(19) United States
(12) Patent Application Publication
(10) Pub. No.: US 2015/0011479 A1
(43) Pub. Date: Jan. 8, 2015

(54) Alpha1 proteinase inhibitor peptides methods and use

(71) Applicant: Cynthia L. Bristow, New York, NY (US)

(72) Inventor: Cynthia L. Bristow, New York, NY (US)

(21) Appl. No.: 14/325,659

(22) Filed: Jul. 8, 2014

Related U.S. Application Data
(63) Continuation of application No. 12/313,889, filed on Nov. 25, 2008.
Application No. 12/313,889 is a continuation-in-part of application No. 11/566,903, filed on Dec. 5, 2006, now abandoned.
(60) Provisional application No. 60/748,137, filed on Dec. 6, 2005.

(51) Int. Cl. A61K 38/57; G01N 33/50; G01N 33/569

(52) U.S. Cl. CPC A61K 38/57; G01N 33/56988; G01N 33/505; G01N 2333/162; G01N 2333/8125; G01N 2333/966; G01N 2500/10; G01N 2500/04; G01N 2800/52
USPC 514/20.4; 435/7.1; 435/5; 506/9

(57) ABSTRACT

The invention is directed to the use of peptides that can bind and block the interaction of α1 proteinase inhibitor (α₁PI) and one or more molecules, for example antibodies to HIV-1 envelope proteins. The invention features methods of activating α₁PI in a cell, methods of treating or preventing a disease or disorder in a subject, for example HIV-1 or AIDS. The invention also features pharmaceutical compositions comprising one or more peptides that block the interaction of α1 α₁PI and one or more molecules. Also included in the invention are kits.

### PRIORITY INFORMATION

This application is a continuation-in-part of U.S. patent application, Ser. No. 11/566,903, filed Dec. 5, 2006, which claims priority from Provisional Application No. 60/748,137 filed Dec. 6, 2005.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### BACKGROUND OF THE INVENTION

Full length active α₁proteinase inhibitor (α₁PI, α₁antitrypsin) is composed of 394 amino acids (aa) having a mass of approximately 55 kDa when filly glycosylated¹. Hepatocytes are the primary source of α₁PI, and in normal, healthy individuals, the range of circulating α₁PI is 20-53 μM between the 5^{th} and 95^{th} percentiles^{2,3}. However, during the acute phase of the inflammatory response, α₁PI may increase as much as 4-fold to 200 μM⁴. There are four common alleles of α₁PI, and these are synthesized and secreted principally by hepatocytes⁵. However, there are more than a hundred genetic variants, some of which produce misfolded molecules that prohibit secretion, e.g. the Z allele, Individuals with this inherited form of α₁PI deficiency, manifest with 10-15% of the normal level of α₁PI in blood¹. Affected individuals, especially males, are notably susceptible to respiratory infections and emphysema, and 80% who survive to adulthood succumb to respiratory failure between the fourth and sixth decades of life⁶. Prevalence is 0.03%, and α₁PI augmentation therapy in affected individuals is the only approved therapeutic application of α₁PI⁵.

Traditionally, α₁PI has been characterized as a proteinase inhibitor which has highest affinity for soluble granule-released elastase (HLE_{G}). Evidence now suggests α₁PI also interacts with cell surface HLE (HLE_{CS})^{7,8}. Both HLE_{CS} and HLE_{G} are synthesized and processed as a single molecular protein; however, HLE is targeted exclusively for the cell surface early in ontogeny and for granule compartmentalization later in ontogeny ^{9,10}. As opposed to its function to inhibit the enzymatic activity of HLE_{G}, α₁PI binding to HLE_{CS} induces cell migration in a manner that does not appear to involve enzymatic activity¹¹. The effect of α₁PI on cell motility is especially profound during migration of stem cells and early progenitor cells. Hematopoiesis begins with stem cell migration from fetal liver through the periphery to the stromal area of hematopoietic tissue, retention, differentiation, and release of maturing progenitor cells back into the periphery. Migration of stem cells to, and myeloid-committed progenitor cells from bone marrow is controlled by HLE_{CS}, the chemokine stromal cell-derived factor-1 (SDF-1, CXCL12), and the SDF-1 receptor CXCR4^{8,12}. Cell migration is dependent on the localization of HLE_{CS} into podia formation at the leading edge of the cell^{8,13}, and podia formation is induced by binding of active α₁PI to HLE_{CS} in a manner that includes co-localization of HLE_{CS} with CD4 and CXCR4⁷.

The current method for therapeutic mobilization of progenitor cells from bone marrow is by the action of G-CSF, and it has been shown that G-CSF mediates this activity by antagonizing CXCR4 and HLE_{CS}¹². G-CSF selectively mobilizes myeloid-committed progenitor cells. The molecular mechanisms that mobilize lymphoid-committed progenitors from hematopoietic tissue are not known. Although α₁PI replacement therapy is effective in producing normal numbers of CD4⁺ lymphocytes, this therapy has many drawbacks including the time involvement and expense.

Accordingly, there remains a need in the art for more effective α₁PI replacement therapy.

### SUMMARY OF THE INVENTION

The invention is directed to the use of peptides, that can bind and block the interaction of α1 proteinase inhibitor (α₁PI) and one or more molecules, for example antibodies to HIV-1 envelope proteins. The invention is based on the finding that the liberation of α₁PI from peptides, in particular antibodies, can be achieved by the use of α₁PI peptides that bind and block such molecules from interacting with full length α₁PI. The invention is based on the finding that these peptides bind the antibodies at a higher affinity than α₁PI. Screening methods and treatment for α₁PI autoimmunity are also provided, resulting from, e.g., HIV-1 infection.

In a first aspect, the invention features a method of activating α1 proteinase inhibitor in a cell comprising contacting the cell with one or more peptides that block an interaction between α1 proteinase inhibitor and one or more molecules, thereby activating α1 proteinase inhibitor.

In another aspect, the invention features a method of restoring α1 proteinase inhibitor activity in a cell comprising contacting the cell with one or more peptides that blocks an interaction between α1 proteinase inhibitor and one or more molecules, thereby restoring α1 proteinase inhibitor activity.

In one embodiment, the activating or restoring α1 proteinase inhibitor results in CD4 lymphocyte renewal.

In another aspect, the invention features a method of increasing CD4 lymphocyte renewal in a cell comprising contacting the cell with one or more peptides that blocks an interaction between α1 proteinase inhibitor and one or more molecules, thereby increasing CD4 lymphocyte renewal.

In one embodiment of any one of the above aspects, the one or more molecules binds and inactivates the α1 proteinase inhibitor.

In another embodiment of any one of the above aspects, the one or more molecules is an antibody.

In another embodiment of any one of the above aspects, the cell is in vivo or in vitro.

In another aspect, the invention features a method of treating or preventing a disease or disorder in a subject comprising administering to the subject one or more peptides that block an interaction between α1 proteinase inhibitor and one or more molecules, thereby treating or preventing the disease or disorder.

In one embodiment, the disease or disorder is selected from the group consisting of atherosclerosis, rheumatoid arthritis, diabetes, allergy, asthma, growth disorder, stem cell therapy, cancer, bacterial infection, viral infection, parasitic infection, and organ transplantation.

In another aspect, the invention features a method of treating a subject suffering from human immunodeficiency virus (HIV-1) comprising administering to the subject one or more peptides that block an interaction between α1 proteinase inhibitor and one or more molecules, thereby treating HIV-1.

In still another aspect, the invention features a method of treating a subject suffering from or susceptible to acquired immune deficiency syndrome (AIDS) comprising administering to the subject one or more peptides that block an interaction between α1 proteinase inhibitor and one or more molecules, thereby treating AIDS.

In one embodiment of any one of the above aspects, the one or more molecules binds to and inactivates the α1 proteinase inhibitor.

In another embodiment of any one of the above aspects, the one or more molecules is an antibody.

In a further embodiment of any one of the above aspects, the method is performed before initiation of HIV-1 antiretroviral therapy.

In one embodiment of any one of the above aspects, the method performed after the initiation of HIV-1 antiretroviral therapy.

In another embodiment of any one of the above aspects, the method is performed concurrently with HIV-1 antiretroviral therapy.

In a further embodiment of any one of the above aspects, the method further comprises monitoring the subject. In a related embodiment, the subject is monitored for a change selected from the group consisting of: active α1 proteinase inhibitor level, CD4 lymphocyte level, changes in HIV-1 RNA copy number and antibodies reactive with α1 proteinase inhibitor.

In another aspect, the invention features a method of screening for one or more agents that blocks the interaction between α1 proteinase inhibitor and one or more molecules that bind and inactivate α1 proteinase inhibitor in a cell comprising producing the peptides, contacting the cell with the one or more agents; and measuring the activation of α1 proteinase inhibitor in the cell compared to a control cell, wherein activation of α1 proteinase inhibitor in the cell identifies an agent that blocks the interaction between α1 proteinase inhibitor and one or more molecules that bind and inactivate α1 proteinase inhibitor.

In one embodiment, the one or more agents is a peptide. In another embodiment, the agents are produced synthetically.

In a further embodiment, the activation of α1 proteinase inhibitor in the cell is measured using one or more assays from the group consisting of, but not limited to inhibition, ability to induce receptor co-capping and cell motility, mobilization of lymphoid-committed progenitor cells, the ability to bind anti-HIV-1 gp120, the ability to facilitate HIV-1 infectivity.

In another embodiments, the one or more molecules is an antibody.

In one embodiment of any one of the above aspects, the molecule is reactive with a viral protein. In a related embodiment, the viral protein is an envelope protein. In a further related embodiment, the envelope protein is HIV-1 gp120.

In another embodiment, the HIV-1 gp120 epitope comprise an amino acid sequence that corresponds to or is complementary to at least a fragment of the amino acid sequence of SEQ ID NO: 3 (GGGDMRDNWRSELYKYKVVK).

In one embodiment of any one of the above aspects, the subject is a mammal.

In another embodiment of any one of the above aspects, the subject is a human.

In one embodiment of any one of the above aspects, the peptide comprises an amino acid sequence that corresponds to or is complementary to at least a fragment of the amino acid sequence of SEQ ID NO: 1.

In another embodiment of any one of the above aspects, the peptide comprises an amino acid sequence that corresponds to or is complementary to residues 357-394 of the amino acid sequence of SEQ ID NO: 1.

In a further embodiment of any one of the above aspects, the peptide comprises an amino acid sequence that corresponds to or is complementary to residues 370-374 of the amino acid sequence of SEQ ID NO: 1.

In still another embodiment of any one of the above aspects, the peptide comprises an amino acid sequence that corresponds to or is complementary to residues 370-385 of the amino acid sequence of SEQ ID NO: 1.

In one embodiment of any one of the above aspects, the peptide comprises an amino acid sequence that corresponds to or is complementary to residues 372-389 of the amino acid sequence of SEQ ID NO: 1.

In another embodiment of any one of the above aspects, the peptide further comprises at least one amino acid substitution. In a related embodiment, the at least one substitution is substitution for a hydrophobic amino acid. In another related embodiment, the hydrophobic amino acid is selected from the group consisting of: isoleucine, leucine, phenylalanine, tyrosine, glycine, threonine, and valine.

In another embodiment, a methionine at position 385 of SEQ ID NO: 1 is substituted with a non-methionine amino acid.

In still another embodiment, the non-methionine amino acid is selected from the group consisting of: glycine, isoleucine, leucine, pheylalanine, threonine and valine.

In another embodiment, a phenylalanine at position 372 of SEQ ID NO; 1 is substituted with a non-phenylalanine amino acid.

In still another embodiment, the non-phenylalanine amino acid is a glycine.

In another embodiment, a leucine at position 373 of SEQ ID NO: 1 is substituted with a non-leucine amino acid.

In still another embodiment, the non-leucine amino acid is a glycine.

In one embodiment, a methionine at position 385 of SEQ ID NO: 1 is substituted with a non-methionine amino acid.

In a further embodiment, the non-methionine amino acid is a valine.

In a related embodiment, the at least one amino acid substitutions is selected from the group consisting of Phe372Gly, Leu373Gly, Leu373Asp, Ile375Arg, Met385Tyr and Met385Val.

In another embodiment, the at least one amino acid substitution comprise four substitutions comprising Phe372Gly, Leu373Asp, Ile375Arg and Met385Tyr.

In another embodiment of any one of the above aspects, the peptides comprise an amino acid sequence that corresponds to or is complementary to SEQ ID NO: 2.

In still another embodiment of any one of the above aspects, the one or more peptides are administered in combination with another agent.

In one embodiment, the agent is a therapeutic agent.

In another embodiment of any one of the above aspects, the peptides are administered at a dose between 1-100 μM.

In another embodiment of any one of the above aspects, the peptides are administered weekly.

In still another embodiment of any one of the above aspects, the peptides are administered monthly.

In another aspect, the invention features a pharmaceutical composition comprising a one or more peptides that block an interaction between α1 proteinase inhibitor and one or more molecules, and a pharmaceutically acceptable carrier.

In one embodiment, the one or more molecules is an antibody.

In another embodiment, the peptide comprises an amino acid sequence that corresponds to or is complementary to at least a fragment of the amino acid sequence of SEQ ID NO: 1.

In one embodiment, the peptide comprises an amino acid sequence that corresponds to or is complementary to residues 357-394 of the amino acid sequence of SEQ ID NO: 1.

In another embodiment, the peptides comprise an amino acid sequence that corresponds to or is complementary to residues 370-374 of the amino acid sequence of SEQ ID NO: 1.

In one embodiment, the peptides comprise an amino acid sequence that corresponds to or is complementary to residues 370-385 of the amino acid sequence of SEQ ID NO: 1.

In another embodiment, the peptides comprise an amino acid sequence that corresponds to or is complementary to residues 372-389 of the amino acid sequence of SEQ ID NO: 1.

In another embodiment of any one of the above aspects, the pharmaceutical composition further comprises at least one amino acid substitution.

In one embodiment, the at least one substitution is substitution for a hydrophobic amino acid.

In another embodiment, the hydrophobic amino acid is selected from the group consisting of: isoleucine, leucine, phenylalanine, tyrosine, glycine, threonine, and valine.

In one embodiment, a methionine at position 385 of SEQ ID NO: 1 is substituted with a non-methionine amino acid.

In another embodiment, the non-methionine amino acid is selected from the group consisting of: glycine, isoleucine, leucine, pheylalanine, threonine and valine.

In one embodiment, a phenylalanine at position 372 of SEQ ID NO: 1 is substituted with a non-phenylalanine amino acid.

In another embodiment, the non-phenylalanine amino acid is a glycine.

In one embodiment, a leucine at position 373 of SEQ ID NO: 1 is substituted with a non-leucine amino acid.

In another embodiment, the non-leucine amino acid is a glycine.

In one embodiment, a methionine at position 385 of SEQ ID NO: 1 is substituted with a non-methionine amino acid.

In another embodiment, the non-methionine amino acid is a valine.

In one embodiment, the at least one amino acid substitutions is selected from the group consisting of: Phe372Gly, Leu373Gly, Leu373Asp, Ile375Arg, Met385Tyr and Met385Val.

In one embodiment the at least one amino acid substitutions comprise Phe372Gly, Leu373Asp, Ile375Arg and Met385Tyr.

In one embodiment, the peptides comprise an amino acid sequence that corresponds to or is complementary to SEQ ID NO: 2.

In one embodiment of any one of the above aspects, the peptide is produced synthetically.

In another aspect the invention features a kit comprising a pharmaceutical composition of any one of the aspects as described herein, and instructions for use.

In another aspect, the invention features a kit for use in any of the methods of any one of the aspects as described herein, and instructions for use.

In other aspects, the methods herein comprise wherein the subject is identified in need of such treatment (e.g. in need α₁PI inhibition).

Other features and advantages of the invention will be apparent from the detailed description, and from the claims.

### BRIEF DESCRIPTION OF DRAWINGS

FIG. 1. Binding of anti-gp120 antibody to human, but not chimpanzee α₁PI. (a) Monoclonal antibody 3F5 binding to α₁PI in sera from 18 healthy humans and 20 healthy chimpanzees was measured in ELISA. Antibody bound (A_{490 nm}) was normalized for the serum α₁PI concentration in each specimen and is represented as A₄₉₀ nm/α₁PI(μM). Binding of 3F5 was 8 to 14-fold greater to human than to chimpanzee α₁PI (p<0.001). Measurements were repeated 6 times using 3F5 and once using monoclonal antibody 1C1. Representative measurements are depicted. Bars represent mean values. (b) The presence of IgG-α₁PI immune complexes in sera (A_{490 nm}) was detected in 11 of 38 HIV-1 infected patients, but not in sera from 9 healthy individuals, 20 healthy chimpanzees, nor in 2 chimpanzees 42 months following HIV-1 inoculation. Serum collected from healthy volunteers into tubes containing clot activating additive were excluded from immune complex analysis because of buffer incompatibility. Measurements were repeated at least 3 times, and representative data are depicted. Bars represent mean values. (c) Active α₁PI concentration in HIV-1 infected patients (median 17 μM) was significantly below normal (median 26 μM, p<0.001). Active α₁PI in sera from 20 healthy chimpanzees (median 35 μM) and 2 chimpanzees post-HIV-1 inoculation median (39 μM) was significantly greater (p<0.02) than from 18 human sera (median 26 μM). Active α₁PI was measured in 8 serial dilutions of each serum sample. (d) Inactive α₁PI concentration in HIV-1 infected patients (median 19 μM) was above normal (median 4 μM, p<0.001). (e) After incubating sera from 5 healthy individuals with monoclonal antibody 3F5, active α₁PI (12±7 μM) was significantly lower than in control sera incubated with medium alone (18±7 μM, p<0.001). Bars represent mean values.

FIG. 2. Corresponding conformation at the 3F5-recognized domains in α₁PI and CD4-complexed HIV-1 gp120. Structures for human α₁PI (1HP7) and CD4-complexed HIV-1 gp120 (1RZJ) from the NCBI Molecular Modeling Database (MMDB) were analyzed using Cn3D software (www.ncbi.nlm.nih.gov/Structure/CN3D/cn3d.shtml). Small carbohydrate structures, depicted in multiple colors, were already associated with 1RZJ in MMDB, and the three associated with 1HP7 were added using Adobe Photoshop, HIV-1 gp120 is depicted from two perspectives (a,b) with green representing two α-helices (aa 21-39 and 306-313). The gp120 peptide immunogen used to raise 1C1 and 3F5 (aa 300-321) is located at the C-terminus of gp120, and the linear segment YKVV (aa 315-318) is depicted in red along with the M-17 and the oligosaccharide-linked NGT (aa 92-94), all of which are within 8 A° of the conformational epitope. The gp120-homologous domain in α₁PI is also located at the C-terminus of the protein, and is depicted from two perspectives (c,d) with violet representing the antiparallel β-sheet strand at the base of the cleft (aa 369-389), and green representing the α-helices that form the mouth of the cleft (aa 28-44 and 259-277). M-385, which distinguishes human from chimpanzee α₁PI, is depicted in red along with GKVV (aa 386-389), the oligosaccharide, and oligosaccharide-linked NST (aa 46-48). The proteinase reactive site M-358, is depicted in yellow for orientation.

FIG. 3. Correlation between CD4⁺ lymphocytes and active α₁PI levels in HIV-1 infected patients. (a) In patients with <500 HIV RNA copies/ml, CD4⁺ lymphocyte levels correlate with active α₁PI (r²=0.927, n=23). CD4⁺ lymphocyte levels also correlate with inactive α₁PI, (r²=0.906, n=23). Patients receiving protease inhibitor therapy are depicted by squares. All other patients are depicted by circles. In 13 patients with >500 HIV RNA copies/ml, no correlation was found to exist between CD4⁺ lymphocyte levels and active α₁PI. (b) IgG-α₁PI immune complexes were correlated with CD4⁺ lymphocyte levels (r²=822, n=8) and with inactive α₁PI (r²=0.988, n=8) in 8 () of the 9 patients with <500 HIV RNA copies/ml. IgG-α₁PI immune complexes were not correlated with CD4⁺ lymphocyte levels or with inactive α₁PI in one patient (◯) who was just above the cutoff with 513 HIV RNA copies/ml.

FIG. 4. Corresponding cyclic increase in CD4⁺ lymphocytes, α₁PI, and viral load in patients treated with α₁PI augmentation. Baseline CD4⁺ lymphocyte levels were determined in patients Alpha, Beta, and Gamma to be 297, 276, and 148 cells/μl at baseline, respectively. Blood was collected prior to infusion, and each data point represents the patient status at 7 days post-infusion such that week 9 represents patient status after the 8^{th} week of treatment. (a) CD4⁺ lymphocytes, CD4/CD8 ratios, and CD4% () vs. the corresponding CD8% (◯) are presented with respect to months of disease diagnosis. Shaded areas represent normal reference ranges for CD4, CD4/CD8 ratio, and CD4%. Black arrows designate initiation of ZEMAIRA treatment. White arrows designate initiation of antiretroviral therapy. (b) Patients Alpha, Beta, Gamma, and PI_{zz}-1 were monitored weekly for changes in blood cell subtypes and serum levels of α₁PI. HIV-1⁺ patients were monitored for changes in HIV RNA. Treatment week 0 represents baseline pre-treatment values. In some instances, blood samples were not acceptable for measuring blood cells, HIV RNA, or α₁PI due to delay in sample delivery or hemolysis, and these are depicted as gaps in the line graphs.

FIG. 5. Sequences of SEQ ID NOs 1-5. SEQ ID NO: 1 is the amino acid sequence corresponding to full length α1PI. SEQ ID NO:2 is the amino acid sequence corresponding to full length α₁PI with amino acid substitutions Phe372Gly, Leu373Asp, Ile375Arg and Met385Tyr. SEQ ID NO: 3 corresponds to the sequence GGGDMRDNWRSELYKYKVVK. SEQ ID NO: 4 is the nucleotide sequence corresponding to full length α₁PI. SEQ ID NO: 5 shows the C-terminal region of α₁PI, defined herein as aa 357-394.

### DETAILED DESCRIPTION OF THE INVENTION

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, the term “control” is meant a standard or reference condition.

As used herein, the term “alpha₁-Proteinase Inhibitor” (α₁PI) is meant to refer to a glycoprotein produced by the liver and secreted into the circulatory system. α₁PI belongs to the Serine Proteinase Inhibitor (Serpin) family of proteolytic inhibitors. This glycoprotein of MW of 50,600 Da consists of a single polypeptide chain containing one cysteine residue and 12-13% carbohydrates of the total molecular weight. α₁PI has three N-glycosylation sites at asparagine residues 46, 83 and 247, which are occupied by mixtures of complex bi- and triantennary glycans. This gives rise to multiple α₁PI isoforms, having isoelectric point in the range of 4.0 to 5.0. The glycan monosaccharides include N-acetylglucosamine, mannose, galactose, fucose and sialic acid. α₁PI serves as a pseudo-substrate for elastase; elastase attacks the reactive center loop of the α₁PI molecule by cleaving the bond between methionine.sub.358-serine.sub.359 residues to form an α₁PI-elastase complex. This complex is rapidly removed from the blood circulation. α₁PI is also referred to as “alpha-1 antitrypsin” (AAT). The term “glycoprotein” as used herein refers to a protein or peptide covalently linked to a carbohydrate. The carbohydrate may be monomeric or composed of oligosaccharides. In certain embodiments, α₁PI is human α₁PI and is encoded by the amino acid sequence set forth by NCBI Accession No. KO1396. In other preferred embodiments,

[symbol]

As used herein, the term “subject” is intended to include vertebrates, preferably a mammal. Mammals include, but are not limited to, humans.

### Methods

Alpha-1 proteinase inhibitor (α₁PI) is a derivative of human plasma belonging to the family of serine proteinase inhibitors. It is a glycoprotein having an average molecular weight of 50,600 daltons, produced by the liver and secreted into the circulatory system. The protein is a single polypeptide chain, to which several oligosaccharide units are covalently bound. α₁PI has a role in controlling tissue destruction by endogenous serine proteinases, and is the most prevalent serine proteinase inhibitor in blood plasma. Among others, α₁PI inhibits trypsin, chymotrypsin, various types of elastases, skin collagenase, renin, urokinase and proteases of polymorphonuclear lymphocytes.

Human α₁PI is shown below in SEQ ID NO: 1, comprising the amino acids set forth in NCBI Accession No. K01396.

SEQ ID NO: 1

| | |
|---|---|
| -24 | MPSSVSWGIL LLAGLCCLVP VSLA |
| 1 | EDPQGDAAQK TDTSHHDQDH PTFNKITPNL AEFAFSLYRQ |
| | LAHQSNSTNI |
| 51 | FFSPVSIATA FAMLSLGTKA DTHDEILEGL NFNLTEIPEA |
| | QIHEGFQELL |
| 101 | RTLNQPDSQL QLTTGNGLFL SEGLKLVDKF LEDVKKLYHS |
| | EAFTVNFGDT |
| 151 | EEAKKQINDY VEKGTQGKIV DLVKELDRDT VFALVNYIFF |
| | KGKWERPFEV |
| 201 | KDTEEEDFHV DQVTTVKVPM MKRLGMFNIQ HCKKLSSWVL |
| | LMKYLGNATA |
| 251 | IFFLPDEGKL QHLENELTHD IITKFLENED RRSASLHLPK |
| | LSITGTYDLK |
| 301 | SVIGQLGITK VFSNGADISO VTEEAPLKIS KAVHKAVLTI |
| | DEKGTEAAGA |
| 351 | MFLEAIPMSI PPEVKFNKPF VFLMIEQNTK SPLFMGKVVN |
| | PTQK |

The known Asn-linked carboxylation sites (denoted in bold underlined letters) are found at aa 46, 83, and 247^{14,15}. The oligosaccharide structure at each site is either tri-antennary or bi-antennary, and the various combinations give the protein a characteristic electrophoretic charge denoted as phenotypic subtypes of the four common genotypic alleles, M1A, M1V, M2, and M3.

The frequencies in US Caucasians of M1A, M1V, M2, and M3 are 0.20-0.23, 0.44-0.49, 0.1-0.11, and 0.14-0.19, respectively, accounting for 95% of this population¹⁵. M1A is thought to be the oldest variant, and the M1V has a single aa substitution, at position 213, Ala to Val. The M3 allele has a single aa difference with M1V, Glu to Asp at position 376. The M2 allele has a single aa difference with M3, Arg to His at position 101.

More than a hundred genotypic alleles have been identified, but except for the S and Z alleles, most of them are exceedingly rare⁵. The S allele, frequency 0.02-0.04, has a single aa substitution at position 264, Glu to Val, and individuals homozygous for this allele manifest 60% normal α₁PI blood levels, but are not a risk for emphysema or other known diseases except in combination with the Z allele^{16,17}. The Z allele, frequency 0.01-0.02, has a single aa substitution at position 342, Glu to Lys, and individuals homozygous for this allele manifest 10% normal α₁PI blood levels, and are at risk for emphysema and autoimmunity.

Functional Properties of α₁PI

In certain embodiments active α₁PI is meant to refer to the fraction of α₁PI in plasma or other fluids that has the capacity to inhibit elastase activity. In other embodiments, inactive α₁PI is meant to refer to the fraction of α₁PI in plasma or other fluids that does not have the capacity to inhibit elastase activity. Active α₁PI may be inactivated by proteolytic cleavage, proteinase complexing, antibody complexing, or oxidation.

The normal role of α₁PI is to regulate the activity of leukocyte elastase, which breaks down foreign proteins present in the lung. When α₁PI is not present in sufficient quantities to inhibit elastase activity, the elastase breaks down lung tissue. In time, this imbalance results in chronic lung tissue damage and emphysema. α₁PI is currently used therapeutically for the treatment of pulmonary emphysema in patients who have a genetic deficiency in α₁PI. Purified α₁PI has been approved for replacement therapy in these patients.

There are three distinct activities of α₁PI that are determined by sites in the C-terminal region of α₁PI, defined herein as aa 357-494 (SEQ ID NO: 5).

PMSI PPEVKFNKPF VFLMIEQNTK SPLFMGKVVN PTQK

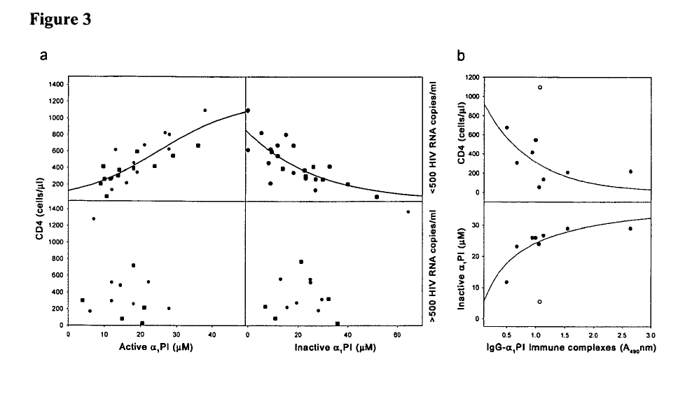

The crystal structure for active α₁PI (1HP7, NIH NCBI Molecular Modeling DataBase mmdbId: 15959) is depicted in FIG. 3 with Met (as 358) and Met (aa 385) designated. The β-sheet formation of the C-terminal region of α₁PI (aa 369-394) is designated. Two α-helix domains (aa 27-44 and 257-280) shield the β-sheet domain in a manner resembling the antigen-binding cleft of the major histocompatibility complex.

The first activity of α₁PI is its well characterized proteinase inhibition which is a property only of active, uncleaved α₁PI. The reactive site for this activity is Met (aa 358) contained in the domain Pro-Met-Ser-Ile-Pro (PMSIP, aa 357-361). Active α₁PI may be inactivated by proteinase complexing, cleavage, or oxidation of Met (aa 358). Interaction at the scissile bond Met-Ser (aa 358-359) may be mediated by many proteinases including HLE_{G}. The two cleavage products of α₁PI may dissociate under some circumstances, but may remain associated in a new, rearranged configuration that may irreversibly incorporate HLE_{G}, but may not incorporate other proteinases, for example metalloproteinases¹⁸.

The tertiary structure for the rearranged α₁PI configuration has not been solved¹⁹; however, X-ray diffraction and kinetic analyses of cleaved α₁PI suggest that the strand SIPPEVKFNKP (aa 359-369) may separate 70 A° from its original position and insert into the β-sheet formation on the opposite face of the molecule (β-sheet A) in a manner that would significantly alter proteinase and receptor recognition²⁰. Thus, four configurations of the C-terminal region of α₁PI are thought to occur (Table 1).

TABLE 1
Functions of the C-terminal region of α₁PI

| | | Lymphoid | Myeloid | anti- | HIV-1 |
|---|---|---|---|---|---|
| | Proteinase | cell | cell | gp120 | entry co- |
| | Inhibition | migration | migration | binding | factor |
| Native | + | + | − | + | + |
| configuration in | | | | | |
| the active α₁PI | | | | | |
| Rearranged | − | − | Unknown | + | − |
| configuration | | | | | |
| in cleaved α₁PI | | | | | |
| Complexed | − | − | + | Unknown | − |
| with HLE | | | | | |
| in cleaved α₁PI | | | | | |
| Independent of | − | Unknown | + | Unknown | Unknown |
| other α₁PI | | | | | |
| cleavage | | | | | |
| products | | | | | |

Because the cleaved configuration of α₁PI lacks proteinase inhibitory activity, in deficient concentrations of active α₁PI, the result is emphysema and respiratory-related infections which are facilitated by the presence of certain environmental factors, cigarette smoke, microbial factors, and inherited mutations that prohibit successful production of active α₁PI.

A second activity of α₁PI is the stimulation of cell migration, and this activity is a property of both cleaved and uncleaved α₁PI. Cleaved α₁PI is recognized by LRP, and stimulates migration of myeloid-lineage cells including neutrophils and monocytic cells²¹. Active, uncleaved α₁PI is recognized by HLE_{CS} and stimulates migration of lymphoid-lineage cells and myeloid-committed progenitor²². Cell migration is initiated by α₁PI-induced co-capping of receptors such as HLE_{CS}, CXCR4, and CD4 into podia formation^{13,23}. In addition to the participation of podia formation during cell migration, this configuration is also the preferred binding site for HIV-1²². The reactive site in α₁PI for this activity is Phe-Val-Phe-Leu-Met (FVFLM, aa 370-374).

A third non-physiologic activity of α₁PI is binding to antibodies reactive with HIV-1 envelope protein gp120, and this activity results in inactivation of α₁PI and blocking of the other two activities described above. The anti-gp120 monoclonal antibodies 1C1 (Repligen, Inc., Cambridge, Mass.) and 3F5 (hybridoma culture supernatant, 0085-P3F5-D5-F8, Dr Larry Arthur NCI-Frederick) were previously shown to be reactive with an epitope near the gp120 C5 domain^{24,25}. The antibody cross-reactive site of human α₁PI is contained in the domain Phe-Leu-Met-Ile-Glu-Gln-Asn-Thr-Lys-Ser-Pro-Leu-Phe-Met-Gly-Lys-Val-Val (FLMIEQNTKSPLFMGKVV, aa 372-389)²⁶ Chimpanzee α₁PI, which differs from human α₁PI by a single amino acid, Val (aa 385), does not bind anti-gp120, consistent with the ability of chimpanzees to resolve HIV-1 infection and regain normal CD4⁺ lymphocyte levels. This suggests that the anti-gp120 cross-reactive site in human α₁PI is determined primarily by the Met residue (aa 385).

α₁PI was also proposed as a treatment for patients homozygous for the defective cystic fibrosis (CF) transmembrane conductance regulator (CFTR) genes, who suffer from recurrent endobronchial infections and sinusitis, malabsorption due to pancreatic deficiency, obstructive hepatobiliary disease and reduced fertility.

There are three products of alpha₁-Proteinase Inhibitor (Human) that are currently FDA approved for treatment. PROLASTIN (on the world wide web at prolastin.com) produced by Talecris Biotherapeutics (on the world wide web at talecris.com), ZEMAIRA (on the world wide web at zemaira.com) produced by ZLB Behring (on the world wide web at zlbbehring.com), and ARALST (on the world wide web at aralast.com) produced by Baxter Healthcare Corp.

The present invention features methods of activating α1 proteinase inhibitor in a cell. In certain embodiments, the method comprises contacting the cell with one or more peptides that block an interaction between α1 proteinase inhibitor and one or more molecules, and thereby activate α1 proteinase inhibitor.

The present invention also features methods of restoring α1 proteinase inhibitor activity in a cell comprising contacting the cell with one or more peptides that blocks an interaction between α1 proteinase inhibitor and one or more molecules, and thereby restoring α1 proteinase inhibitor activity.

In certain cases, activating or restoring α1 proteinase inhibitor results in CD4 lymphocyte renewal.

Accordingly, the invention features a method of increasing CD4 lymphocyte renewal in a cell comprising contacting the cell with one or more peptides that blocks an interaction between α1 proteinase inhibitor and one or more molecules, thereby increasing CD4 lymphocyte renewal.

The cell may preferably be in vitro, in certain embodiments. In other embodiments, the cell can be in vivo.

In embodiment of any of the methods as described herein the one or more molecules binds and inactivates the α1 proteinase inhibitor.

In another embodiment any of the methods as described herein, the one or more molecules is an antibody.

Evidence now shows that during HIV-1 disease, antibodies with specificity for HIV-1 envelope proteins also bind host proteins²⁷. The specificity of one such antibody also binds and inactivates α₁PI (US2008/0009442 incorporated by reference in its entirety herein), and this produces functional deficiency of α₁PI in HIV-1 infected individuals. Such deficiency prevents CD4⁺ lymphocyte renewal and leads to AIDS. Therapeutic α₁PI infusion reinstates normal CD4⁺ lymphocyte renewal (US2008/0009442 and herein).

Accordingly, the invention also features methods for treating diseases. In one aspect, the invention features a method of treating or preventing a disease or disorder in a subject comprising administering to the subject one or more peptides that block an interaction between α1 proteinase inhibitor and one or more molecules, and thereby treating or preventing the disease or disorder.

In other embodiments, the one or more molecules is preferably an antibody.

In certain preferred embodiments of any of the methods described herein the molecule is reactive with a viral protein. Preferably, the viral protein is an envelope protein. Even more preferably, in certain examples, the envelope protein is HIV-1 gp120.

Accordingly, the invention features methods of treating or preventing a disease or disorder in a subject comprising administering to the subject one or more peptides that block an interaction between α1 proteinase inhibitor and one or more molecules, where the molecule is HIV-1 gp120, thereby treating or preventing the disease or disorder.

In further examples, the HIV-1 gp120 epitope comprises an amino acid sequence that corresponds to or is complementary to at least a fragment of the amino acid sequence of SEQ ID NO: 3 (GGGDMRDNWRSELYKYKVVK).

The disease or disorder that can be treated can be selected from any number of diseases or disorders, for example those diseases where increasing CD4 lymphocyte renewal is beneficial.

In one embodiment, the disease or disorder is selected from the group consisting of, but not limited to, atherosclerosis, rheumatoid arthritis, diabetes, allergy, asthma, growth disorder, stem cell therapy, cancer, bacterial infection, viral infection, parasitic infection, and organ transplantation.

In other aspects, the invention features a method of treating a subject suffering from human immunodeficiency virus (HIV-1) or a method of treating a subject suffering from or susceptible to acquired immune deficiency syndrome (AIDS), where the methods comprise administering to the subject one or more peptides that block an interaction between α1 proteinase inhibitor and one or more molecules, thereby treating HIV-1.

Regarding the use of the method for the treatment of a subject suffering from human immunodeficiency virus (HIV-1), in certain embodiments, the method is performed before initiation of HIV-1 antiretroviral therapy. In other embodiments, the method is performed after the initiation of HIV-1 antiretroviral therapy. In still other embodiments, the the method is performed concurrently with HIV-1 antiretroviral therapy.

In the methods as described, in certain preferred embodiments, the one or more peptides are administered in combination with another agent. In certain cases, it is preferred that the agent is a therapeutic agent.

The agent may, in other examples, be an antiretroviral therapeutic.

Antiretroviral drugs inhibit the replication of HIV. When antiretroviral drugs are given in combination, HIV replication and immune deterioration can be delayed, and survival and quality of life improved. Taking two or more antiretroviral drugs at a time is called combination therapy. Taking a combination of three or more anti-HIV drugs is sometimes referred to as Highly Active Antiretroviral Therapy (HAART). There are over 20 approved antiretroviral drugs although all are licensed or available in every country. Antiretroviral drug classes include: Nueleoside/Nucleotide Reverse Transcriptase Inhibitors (NRTI), Non-Nucleoside Reverse Transcriptase Inhibitors (NNRTI), Protease Inhibitors, Fusion or Entry Inhibitors, and Integrase Inhibitors.

For example, a common drug combination given to those beginning treatment consists of two NRTIs combined with either an NNRTI or a “boosted” protease inhibitor. Ritonavir (in small doses) is most commonly used as the booster; it enhances the effects of other protease inhibitors so they can be given in lower doses. An example of a common antiretroviral combination is the two NRTIs zidovudine and lamivudine, combined with the NNRTI efavirenz.

The invention also features methods of monitoring the subject.

For example, the subject can be monitored for a change selected from the group consisting of, but not limited to, active α1 proteinase inhibitor level, CD4 lymphocyte level, changes in HIV-1 RNA copy number and antibodies reactive with α1 proteinase inhibitor.

### Screening

One embodiment of the invention encompasses a method of identifying one or more agents that blocks the interaction between α1 proteinase inhibitor and one or more molecules that bind and inactivate α1 proteinase inhibitor in a cell. Accordingly, compounds or peptides that modulate the interaction between α1 proteinase inhibitor and one or more molecules that bind and inactivate α1 proteinase inhibitor, polypeptide, variant, or portion thereof, are useful in the methods of the invention for the treatment or prevention of a disease or disorder, and in particular, for the treatment of HIV-1.

In preferred embodiments, the one or more molecules are antibodies. Thus the methods identify peptides that bind to antibodies at a higher affinity than α₁PI. In preferred embodiments, the invention features peptides that bind to antibodies at preferred epitopes and then are further screened for other activities, for example elastase inhibition, ability to induce receptor co-capping and cell motility, mobilization of lymphoid-committed progenitor cells, the ability to bind anti-HIV-1 gp120, the ability to facilitate HIV-1 infectivity. Preferably, peptides are identified that bind to antibodies that do not have these other effects and do not have toxic effects. It is a feature of the invention thought that, for example, a peptide that does exhibit any one of the activities described herein (e.g. elastase inhibition), or another activity not described, but that is useful, will have uses in other therapies.

Any number of methods are available for carrying out screening assays to identify such compounds. In one approach, the method comprises producing or obtaining the agents, contacting the cell with the agents; and measuring the activation of α1 proteinase inhibitor in the cell compared to a control cell; wherein activation of α1 proteinase inhibitor in the cell identifies an agent that blocks the interaction between α1 proteinase inhibitor and one or more molecules that bind and inactivate α1 proteinase inhibitor.

In another approach, candidate compounds are identified that specifically bind to and alter the activity of a polypeptide of the invention (e.g., activation of α1 proteinase inhibitor in the cell). Methods of assaying such biological activities are known in the art. The efficacy of such a candidate compound or peptide is dependent upon its ability to modulate the interaction between α1 proteinase inhibitor and one or more molecules that bind and inactivate α1 proteinase inhibitor.

Potential agents that may be identified include peptides, peptide mimetics, polypeptides, organic molecules, nucleic acid molecules (e.g., double-stranded RNAs, siRNAs, antisense polynucleotides), and antibodies that bind to a nucleic acid sequence or polypeptide of the invention and thereby inhibit or extinguish its activity. Potential antagonists also include small molecules that that blocks the interaction between a α1 proteinase inhibitor and one or more molecules that bind and inactivate α1 proteinase inhibitor. Small molecules of the invention preferably have a molecular weight below 2,000 daltons, more preferably between 300 and 1,000 daltons, and still more preferably between 400 and 700 daltons. It is preferred that these small molecules are organic molecules.

Any in vivo protein interaction detection system, for example, any two-hybrid assay may be utilized to identify compounds that blocks the interaction between α1 proteinase inhibitor and one or more molecules that bind and inactivate α1 proteinase inhibitor (and, for example, to increase α1 proteinase inhibitor activity). Interacting compounds isolated by this method (or any other appropriate method) may, if desired, be further purified (e.g., by high performance liquid chromatography). Compounds isolated by any approach described herein may be used as therapeutics to treat a disease or disorder, for example HIV-1, in a human patient.

The invention also includes novel compounds identified by the above-described screening assays. Optionally, such compounds are characterized in one or more appropriate animal models to determine the efficacy of the compound for the treatment of a disease or disorder, for example, HIV-1. Desirably, characterization in an animal model can also be used to determine the toxicity, side effects, or mechanism of action of treatment with such a compound. Furthermore, novel compounds identified in any of the above-described screening assays may be used for the treatment of a disease or disorder, for example HIV-1, in a subject. Such compounds are useful alone or in combination with other conventional therapies known in the art.

According to preferred embodiments of the invention, and as described herein, the desired α₁PI peptides for treating a disease or disorder are those that bind α₁PI-reactive antibodies, but do not functionally interfere with the physiologic activity of α₁PI.

Peptides derived from α₁PI are selected for use in treatment of specific blood cell diseases by determining their capacity in vitro and in vivo to influence the following functions in the following assays: (1) elastase inhibition, for example as described by U.S. Pat. No. 6,887,678, incorporated by reference in its entirety herein. (2) ability to induce receptor co-capping and cell motility. (3) Mobilization of lymphoid-committed progenitor cells. (4) Ability to bind anti-HIV-1 gp120. (5) Ability to facilitate HIV-1 infectivity.

To determine the influence of α₁PI peptide treatment on elastase inhibitory capacity, individuals are monitored weekly for levels of active and inactive α₁PI in blood³⁹ (U.S. Pat. No. 6,887,678). Briefly, a constant amount of active site-titrated PPE is allowed to incubate with serial dilutions of serum for 2 min at 37° C. after which a PPE substrate is added. Determination of the molecules of substrate cleaved by residual, uninhibited PPE is used to calculate the molecules of active and inactive α₁PI in blood.

To determine the influence of α₁PI peptide treatment on inducing changes in levels of blood cell populations, treated individuals are monitored weekly for changes in complete blood count and differential, as well as for changes in specific subsets of blood cells such as DC4⁺ cells and HLE_{CS}⁺ cells using flow cytometry^{26,46} (U.S. Pat. No. 6,858,400). Briefly, 100 μl of whole blood is incubated with a panel of fluorescently-labeled monoclonal antibodies approved by the FDA for medical diagnostics. These antibodies are selected to specifically recognize the cell receptors that uniquely identify the cell population of interest. Identification and enumeration of the cells in blood that are bound to the monoclonal antibodies is performed using flow cytometry. To determine the influence of treatment on disease progression, individuals are monitored for the specific pathologic determinants of disease which are well known in the art for the various indications in HIV-1 disease. For example, in HIV-1 disease, individuals are monitored for changes in CD4⁺ lymphocyte levels and HIV levels^{26,46} as well as for

[symbol]

Test Compounds and Extracts

In certain embodiments, compounds capable blocks the interaction between α1 proteinase inhibitor and one or more molecules that bind and inactivate α1 proteinase inhibitor are identified from large libraries of either natural product or synthetic (or semi-synthetic) extracts or chemical libraries according to methods known in the art. Methods for making siRNAs are known in the art. Numerous methods are also available for generating random or directed synthesis (e.g., semi-synthesis or total synthesis) of any number of chemical compounds, including but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK). Harbor Branch Oceangraphics Institute (Ft. Pierce, Fla.), and PharmaMar, U.S.A. (Cambridge, Mass.).

In one embodiment, test compounds of the invention are present in any combinatorial library known in the art, including: biological libraries; peptide libraries (libraries of molecules having the functionalities of peptides, but with a novel, non-peptide backbone which are resistant to enzymatic degradation but which nevertheless remain bioactive; see, e.g., Zuckermann, R. N. et al., J. Med. Chem. 37:2673-85, 1994); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, Anticancer Drug Des. 12:145, 1997).

Examples of methods for the synthesis of molecular libraries can be found in the art, for example in: DeWitt et al., Proc. Natl. Acad. Sci. U.S.A. 90:6909, 1993; Erb et al., Proc. Natl. Acad. Sci. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994.

Libraries of compounds may be presented in solution (e.g., Houghten Biotechniques 13:412-421, 1992) or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their anti-neoplastic activity should be employed whenever possible.

In an embodiment of the invention, a high thoroughput approach can be used to screen different chemicals for their potency to affect α1 proteinase inhibitor activity.

Those skilled in the field of drug discovery and development will understand that the precise source of a compound or test extract is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds.

When a crude extract is found to be of interest, e.g. to block the interaction between α1 proteinase inhibitor and one or more molecules that bind and inactivate α1 proteinase inhibitor (for example, to increase α1 proteinase inhibitor activity), further fractionation of the positive lead extract is necessary to isolate chemical constituents responsible for the observed effect. Thus, the goal of the extraction, fractionation, and purification process is the careful characterization and identification of a chemical entity within the crude extract having anti-neoplastic activity. Methods of fractionation and purification of such heterogeneous extracts are known in the art. If desired, chemical modification can be carried out according to methods known in the art.

### Pharmaceutical Compositions

The one or more peptides that block an interaction between α1 proteinase inhibitor and one or more molecules of the present invention can be administered as part of a pharmaceutical composition.

Accordingly, the invention features a pharmaceutical composition comprising a one or more peptides that block an interaction between α1 proteinase inhibitor and one or more molecules, and a pharmaceutically acceptable carrier. The molecules, in certain preferred embodiments, can be antibodies.

Such a pharmaceutical composition can include any standard physiologically and/or pharmaceutically acceptable carrier known in the art (e.g., liposomes/cationic lipids/creams). See Remington: The Science and Practice of Pharmacy, Alfonso R. Gennaro, editor, 20th ed. Lippincott Williams & Wilkins: Philadelphia, Pa., 2000. The compositions should be sterile and contain a therapeutically effective amount of therapeutic agent in a unit of weight or volume suitable for administration to a patient. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism.

The one or more peptides that block an interaction between α1 proteinase inhibitor and one or more molecules can be administered via a variety of routes including, but not limited to topical, transdermal, oral, subcutaneous and the like via standard medical practices.

The one or more peptides that block an interaction between α1 proteinase inhibitor and one or more molecules of the instant invention (i.e., antibodies) can be administered alone or admixed together with a suitably acceptable carrier to provide even greater therapeutic effect. Moreover, the one or more peptides that block an interaction between α1 proteinase inhibitor and one or more molecules of this invention can be combined with other agents to provide further therapeutic benefit, e.g. synergistic therapeutic properties.

A peptide of the invention can be used alone or in combination with other agents for the manufacture of a medicament for use in the treatment of wounds of an animal, preferably a human. Alternatively, a portion of an alpha-1 proteinase inhibitor can be used for treating a disease or condition associated with the liver. In accordance with such treatment, an effective amount of at least a portion of an alpha-1 proteinase inhibitor is administered to an animal or human patient so that a disease or condition associated with the liver is treated. Subjects who could benefit from such treatment include those with liver diseases or conditions including, but not limited to, alpha-1 proteinase deficiency combined with liver dysfunctions such as cirrhosis or hepatitis.

One embodiment of the instant invention embraces at least a portion of one or more peptides that block an interaction between α1 proteinase inhibitor and one or more molecules. As used in the context of the instant invention, at least a portion of one or more peptides that block an interaction between α1 proteinase inhibitor and one or more molecules is intended to mean a portion of a peptide that still retains the ability to block an interaction between α1 proteinase inhibitor and one or more molecules. Accordingly, at least a portion can be 3, 4, 5, 6, 7, 8, 9, 10, 11, 12, 13, 14, 15, 20, 25, 30, 35, 40, 45, 50, 55, 60, 65, 70, 75, 80, 90, 100, 110, 120, 140, 150, 175, 200, 225, 250, 275, 300 or more amino acids.

In certain cases, the molecule is reactive with a viral protein, for example an envelope protein. An envelope protein, preferably, can be HIV-1 gp120.

In further preferred embodiments, the HIV-1 gp120 comprises an epitope that corresponds to or is complementary to at least a fragment of the amino acid sequence of SEQ ID NO: 3, shown below:

SEQ ID NO 3
GGGDMRDNWRSELYKYKVVK

The peptides disclosed herein can be modified by deletion, substitution or addition of at least one amino acid residue of the sequence. A modified or variant polypeptide and reference polypeptide may differ in amino acid sequence by one or more substitutions, additions, deletions, truncations which may be present in any combination. Among preferred variants are those that vary from a reference polypeptide by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and asparatic acid; c) asparagine and glutamine d) arginine and lysine; e) isoleucine, leucine, methionine and valine and f) phenylalanine, tyrosine and tryptophan. Desirably a variant retains the same biological function and activity as the reference polypeptide from which it varies.

A functionally equivalent polypeptide according to the invention is a variant wherein one or more amino acid residues are substituted with conserved amino acid residues, or one in which one or more amino acid residues includes a substituent group. Conservative substitutions are the replacements, one for another, among the aliphatic amino acids Ala, Val, Leu and Ile; interchange of the hydroxl residues Ser and Thr; exchange of the acidic residues Asp and Glu; substitution between amide residues Asn and Gln; exchange of the basic residues Lys and Arg; and replacements among aromatic residues Phe and Tyr.

In addition, the invention embraces polypeptide sequences having at least 75% identity with the polypeptide sequences as herein disclosed, or fragments and functionally equivalent polypeptides thereof. In one embodiment, the polypeptides have at least 85% identity, more preferably at least 90% identity, even more preferably at least 95% identity, still more preferably at least 97% identity, and most preferably at least 99% identity with the amino acid sequences illustrated herein.

In certain examples, the peptide comprises an amino acid sequence that corresponds to or is complementary to at least a fragment of the amino acid sequence of SEQ ID NO: 1.

In certain examples, the peptide comprises an amino acid sequence that corresponds to or is complementary to residues 357-394 of the amino acid sequence of SEQ ID NO: 1.

In certain examples, the peptides comprise an amino acid sequence that corresponds to or is complementary to residues 370-374 of the amino acid sequence of SEQ ID NO: 1.

In certain examples, the peptides comprise an amino acid sequence that corresponds to or is complementary to residues 370-385 of the amino acid sequence of SEQ ID NO: 1.

In certain examples, the peptides comprise an amino acid sequence that corresponds to or is complementary to residues 372-389 of the amino acid sequence of SEQ ID NO: 1.

Further, the peptides may comprise at least one amino acid substitution. In certain preferred examples the at least one substitution is substitution for a hydrophobic amino acid. Preferably, the hydrophobic amino acid is selected from the group consisting of isoleucine, leucine, phenylalanine, tyrosine, glycine, threonine, and valine.

In preferred embodiments, a methionine at position 385 of SEQ ID NO: 1 is substituted with a non-methionine amino acid. Preferably, the non-methionine amino acid is selected from the group consisting of: glycine, isoleucine, leucine, pheylalanine, threonine and valine.

In certain examples, a phenylalanine at position 372 of SEQ ID NO; 1 is substituted with a non-phenylalanine amino acid. In other preferred embodiments, the non-phenylalanine amino acid is a glycine.

In certain examples, a leucine at position 373 of SEQ ID NO: 1 is substituted with a non-leucine amino acid. In other preferred embodiments, the non-leucine amino acid is a glycine.

In certain examples, a methionine at position 385 of SEQ ID NO: 1 is substituted with a non-methionine amino acid. In other preferred embodiments, the non-methionine amino acid is a valine.

The at least one amino acid substitutions, in preferred examples, is selected from the group consisting of Phe372Gly, Leu373Gly, Leu373Asp, Ile375Arg, Met385Tyr and Met385Val.

The at least one amino acid substitutions, in other preferred examples, comprise Phe372Gly, Leu373Asp, Ile375Arg and Met385Tyr.

In preferred embodiments, the peptides comprise an amino acid sequence that corresponds to or is complementary to SEQ ID NO: 2.

In preferred embodiments, the peptides are synthetically produced.

Peptides of the instant invention can be produced by recombinant DNA technology or chemically synthesized, or produced by a combination thereof. A protein composition produced by recombinant DNA technology is generally expressed from a nucleic acid encoding the protein. Such a nucleic acid can be isolated by convention methodologies such as restriction enzyme-based cloning. For example, DNA fragments coding for the different protein or peptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. Alternatively, the isolated nucleic acid molecule can be synthesized by conventional techniques including automated DNA synthesis or polymerase chain reaction (PCR) amplification. PCR amplification of gene fragments can be carried out using anchor primers which give rise to complementary overhangs between two consecutive gene fragments which are subsequently annealed and reamplified to generate a chimeric gene sequence (see, e.g., Current Protocols in Molecular Biology, eds. Ausubel, et al. John Wiley & Sons, 1992).

Recombinant production of a desired protein typically involves directly expressing the desired protein from a recombinant expression vector or expressing the desired protein with a heterologous protein sequence such as a tag or a signal sequence to facilitate purification or secretion of the desired protein from a host cell. In certain host cells (e.g., mammalian host cells), expression and/or secretion of a desired protein can be increased through use of a heterologous signal sequence. Such tags include, but are not limited to a his-tag or FLAG®-tag.

A recombinant expression vector generally harbors nucleic acids encoding the desired protein in a form suitable for expression, i.e., the recombinant expression vector includes one or more regulatory sequences operatively-linked to the nucleic acid to be expressed. Expression vector and recombinant expression vector are used interchangeably herein, and in the context of a recombinant expression vector, operatively-linked is intended to mean that the nucleic acid of interest is linked to the regulatory sequence(s) in a manner which allows for expression of the nucleic acid (e.g., in an in vitro transcription/translation system or in a host cell). A regulatory sequence is intended to include promoters, enhancers and other expression control elements (e.g., polyadenylation signals). Such regulatory sequences are described, for example, in Goeddel (1990) Methods Enzymol. 185:3-7. Regulatory sequences include those which direct constitutive expression of a nucleic acid in many types of host cells and those which direct expression of the nucleic acid only in certain host cells (e.g., tissue-specific regulatory sequences). It will be appreciated by one of skill in the art that the design of the expression vector depends on such factors as the choice of the host cell to be transformed, the level of expression of the desired protein, and the like.

A recombinant expression vector can be designed for expression of a desired protein in prokaryotic or eukaryotic cells. For example a protein composition of the instant invention can be expressed in bacterial cells such as E. coli, insect cells (using baculovirus expression vectors), yeast cells or mammalian cells. Suitable host cells are discussed further in Goeddel (1990) supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Examples of suitable inducible E. coli expression vectors include pTrc (Amann, et al. (1988) Gene 69:301-315) and pET 1d (Studier, et al. (1990) Methods Enzymol. 185:60-89). Target gene expression from the pTrc vector relies on host RNA polymerase transcription from a hybrid trp-lac fusion promoter. Target gene expression from the pET lid vector relies on transcription from a T7 gn10-lac fusion promoter mediated by a coexpressed viral RNA polymerase (T7 gn1). This viral polymerase is supplied by host strains BL21 (DE3) or HMS174(DE3) from a resident prophage harboring a T7 gn1 gene under the transcriptional control of the lacUV 5 promoter.

A yeast expression vector also encompassed within the scope of the invention. Examples of vectors for expression in yeast such as Saccharomyces cerevisiae include pYepSec 1 (Baldari, et al. (1987) EMBO J. 6:229-234), pMFa (Kurjan and Herskowitz (1982) Cell 30:933-943), pJRY88 (Schultz, et al. (1987) Gene 54:113-123), pYES2 (INVITROGENT™ Corp., San Diego, Calif.), and picZ (INVITROGEN™ Corp., San Diego, Calif.).

Alternatively, a protein composition of the invention can be expressed in insect cells as exemplified herein using baculovirus expression vectors. Baculovirus vectors available for expression of proteins in cultured insect cells (e.g., Sf9 cells) include the pAc series (Smith, et al. (1983) Mol. Cell Biol. 3:2156-2165) and the pVL series (Lucklow and Summers (1989) Virology 170:31-39) of vectors.

Recombinant expression vectors in which the nucleic acid of interest is homologously recombined into a specific site of the host cell's genome are also contemplated. The terms host cell and recombinant host cell are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Expression vector DNA can be introduced into prokaryotic or eukaryotic cells via conventional transformation or transfection techniques. As used herein, the terms transformation and transfection are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (1989) supra, and other laboratory manuals.

To identify and select transformed or transfected host cells, a gene that encodes a selectable marker (e.g., resistance to antibiotics) is generally introduced into the host cells along with the nucleic acid of interest. Suitable selectable markers include those which confer resistance to drugs, such as G418, hygromycin and methotrexate. Nucleic acids encoding a selectable marker can be introduced into a host cell on the same vector as that encoding the protein of interest or can be introduced on a separate vector. Cells stably transformed or transfected with the introduced nucleic acid can be identified by drug selection (e.g., cells that have incorporated the selectable marker gene will survive, while the other cells die).

Once produced, the desired protein or peptide is either recovered as a secreted protein or from host cell lysates, when directly expressed without a secretory signal. Purification of the protein composition from recombinant cell proteins can be carried out by centrifuging the culture medium or lysate to remove particulate cell debris and purifying the protein composition by, e.g., fractionation on immunoaffinity or ion-exchange columns, ethanol precipitation, chitin column chromatography, reverse phase HPLC, chromatography on silica or on a anion-exchange resin such as DEAE, chromatofocusing, SDS-PAGE, ammonium sulfate precipitation, gel filtration, or ligand affinity chromatography (e.g., Ni²⁺-agarose chromatography).

In addition to recombinant production, the protein composition can be produced by direct peptide synthesis using solid-phase techniques (Merrifield (1963) J. Am. Chem. Soc. 85:2149-2154). Protein synthesis can be performed using manual techniques or by automation. Automated synthesis can be achieved, for example, using Applied Biosystems 431A Peptide Synthesizer (Perkin Elmer, Boston, Mass.). Various fragments of the protein composition can be chemically-synthesized separately and combined using chemical methods to produce the full-length molecule. A number of cross-linkers are well-known in the art, including homo or hetero-bifunctional cross-linkers, such as BMH,, SPDP, etc. Chemical methods for cross-linking molecules to the amino- or carboxy-terminus of a protein are reviewed by Oxford (1992) In: Protein Engineering—A Practical Approach, Rees, et al., eds., Oxford University Press.

In preferred embodiments, in addition to α₁PI peptides synthesized from individual amino acids, recombinant α₁PI peptides will be used for blocking α₁PI-reactive antibodies.

In certain preferred examples, any method known in the art may be used for producing recombinant α₁PI peptides according to the invention. Two preferred methods are briefly described below for producing recombinant α₁PI peptides; one allows expression of α₁PI peptides in rice cells and the other allows bacterial expression. The cDNA encoding human α₁PI is obtained from a human cDNA bank by and amplification of the fragment in accession number K01396 using two PCR primers:

N-terminal primer
5′ GAGGATCCCCAGGGAGATGCTGCCCAGAA 3′
and
C-terminal primer
5′ CGCGCTCGAGTTATTTTTGGGTGGGATTCACCAC 3′
as previously described ²⁸⁻³⁰.

For expression in rice cells, expression cassettes are prepared by using a 1.1 kb NheI-PstI fragment, derived from plAS1.5 is cloned into the vector pGEM5zf-(Promega, Madison, Wis.): ApaI, AatII, SphI, NcoI, SStII, EcoRV, SpeI, NotI, PstI, SalI, NdeI, SacI, MluI, NsiI at the SpeI and PstI sites to form pGEM5zf-(3D/NheI-PstI). The GEM5zf-(3D/NheI-PstI) is digested with PstI and SacI and ligated in two nonkinased 30 mers with the complementary sequences 5′ GCTTG ACCTG TAACT CGGGC CAGGC GAGCT 3′ and 5′ CGCCT AGCCC GAGTT ACAGG TCAAG CAGCT 3′ to form p3DProSig. A 5-kb BamHI-KpnI fragment from lambda clone λOSgl A is used as a terminator. Hygromycin resistance is obtained from the 3-kb BamHI fragment containing the 35S promoter-Hph-NOS of the plasmid pMON410.

Microprojectile bombardment is applied for transforming a Japonica rice variety TP309. The bombarded calli are then transferred to NB medium containing 50 mg/l hygromycin and incubated in the dark at 25° C. for 10±14 days. Rice cells are cultured at 28° C. (dark) using a shaker with rotation speed 115 rpm in the AA(+sucrose) media. The medium is changed every 5 days to maintain cell lines. AA(−sucrose) is used for α₁PI expression. A bioreactor is used for 2-1-scale culture. The reactor is operated at 28° C. (dark) at agitation speed 30±50 rpm with aeration rate 100 ml/min. During the growth phase (10 days), the pH of the media is controlled at pH 5.7, while in the production phase the PH is 5.7±6.3 (un-controlled).

Recombinant α₁PI peptides are purified using anti-human α₁PI antibody (Enzyme Research Laboratories, South Bend, Ind.) or anti-HIV-1 gp120 (Science Applications International Corporation, Frederick, Md.) immobilized to CNBr-activated Sepharose 4B with a concentration of 1.5 mg/ml gel. The gel (3.5 ml) is packed in a column (inner diameter 1.26 cm), and equilibrated with 50 mM Tris-HCl buffer (pH 7.6). Crude medium is applied to the column at 1.0 ml/min. Absorbance at 280 nm is monitored at the outlet of the column. After washing with the equilibrium buffer, α₁PI is eluted with 0.1N HCl solution. A peak fraction is collected, and its pH is immediately adjusted with 1M Tris-HCl buffer (pH 8.0). These methods yield an estimated 5.7 mg α₁PI peptide/g dry cell.

Alternatively, the α₁PI peptides cDNA are expressed in Escherichia coli strain BL21 transformed with PDS56α₁PI/hf (Invitrogen, Carlsbard, Calif.). Protein expression is induced by addition of 1 mM isopropyl b-D-thiogalactoside, and cultures are grown overnight at 31° C. The cells are washed in metal-chelation chromatography binding buffer (5 mM imidazole/0.5M. NaCl/20 mM Tris, pH 7.9) and disrupted by cavitation. The clarified and filtered supernatants containing soluble α₁PI peptides are applied to a Ni²⁺-agarose column, and bound peptides are eluted with 100 mM EDTA. The eluates are adjusted to 35M NaCl and applied to a phenyl-Sepharose column. The bound α₁PI peptide/hf is eluted with 20 mM Bis-Tris, pH 7.0 and concentrated (4 mg/ml final) by diafiltration in the same buffer.

Genetic Modification of α₁PI Peptides

Recombinant α₁PI peptides are expressed according to the procedures described herein. Wild-type human α₁PI peptides are modified genetically to diminish or enhance sequence-specific reactive sites. For example, in HIV-1 disease, therapeutic α₁PI peptides maintain binding to anti-gp120, but do not interfere with full sequence α₁PI in its activities to inhibit soluble HLE_{G} and to induce cell migration.

The genetic modifications of interest are described herein. Site-directed mutagenesis of active α1PI is performed using standard procedures^{31,32}. The DNA sequence encoding the human α1PI signal peptide in pDS56α₁PI/hf is replaced with sequences encoding the epitope (FLAG)-tag by insertion of the annealed complimentary oligos 5′ CTAGAGGATCCCATGGACTACAAGGACGACGATGACAAGGAA 3′ and 5′GATCTTCCTTGTCATCGTCGTCCTTGTAGTCCATGGGATCCT 3′. The resulting cDNA is subcloned into pDS56-6His to generate pDS56α1PI/hf. To generate pDS56α1PI/hf carrying an amino acid substitution, the DNA sequences encoding the wild-type amino acid are replaced by the complimentary oligos coding for the amino acids described herein. The resulting ORFs directed cytosolic expression of the recombinant proteins initiating with a Met followed by the His and FLAG tags and the mature sequences of mutant α1PI.

In certain preferred embodiments, modification within the domain that determines cell migration (FVFLM, aa 370-374) is prepared by site-directed mutagenesis of specific amino acids:

### Phe (aa 370) to Ile, Leu, Val, Tyr, or Gly.

### Val (aa 371) to Phe, Leu, Ile, or Gly.

### Phe (aa 372) to Ile, Leu, Val, Tyr or Gly.

### Leu (aa 373) to Ile, Val, Phe, or Gly.

### Met (aa 374) to Phe, Thr, Ile, Leu, Val, or Gly.

Modification within the domain that determines HIV-1 gp120 antibody recognition is prepared by site-directed mutagenesis of Met (aa 385) to Phe, Thr, Ile, Leu, Val, or Gly.

In certain preferred embodiments, peptides will be prepared by Fmoc solid-phase synthesis as previously described³³ and subsequently purified by reversed-phase chromatography. Identity and homogeneity of the products will be analyzed by reversed-phase HPLC, capillary zone electrophoresis, electrospray mass spectrometry, and sequence analysis. After proteolytic modification, the C-terminal α₁PI domain acquires attributes that allow interaction with the LDL receptor-related protein (LRP)³⁴, the VLDL receptor³⁵, and other receptors that recognize a pentapeptide sequence FVFLM (aa 370-374)²¹ in a manner that produces, chemotaxis of neutrophils, increased LDL binding to monocytes, upregulated LDL receptors, increased cytokine production, and α₁PI synthesis^{23,36,36}. It has been shown that fibrillar aggregates of the C-terminal fragment of α₁PI facilitate uptake of LDL by LRP on the hepatolastoma cell line HepG2³⁷, and these fragments participate in atherosclerosis³⁸.

According to preferred embodiments of the invention, and as described herein, the desired α₁PI peptides for treating a disease or disorder are those that bind α₁PI-reactive antibodies, but do not functionally interfere with the physiologic activity of α₁PI.

Peptides derived from α₁PI are selected for use in treatment of specific blood cell diseases by determining their capacity in vitro and in vivo to influence the following functions in the following assays:

Inhibit elastase: The procedures for measuring the capacity of α₁PI to inhibit soluble forms of porcine pancreatic elastase (PPE) or HLE_{G} are well established (U.S. Pat. No. 6,887,678)³⁹. Briefly, PPE is incubated for 2 min with α₁PI, and to this mixture is added, the elastase substrate succinyl-L-Ala-L-Ala-L-Ala-p-nitroanilide (SA³NA). Results are detected by measuring the color change at 405 nm.

In complex mixtures, α₁PI competes for binding to PPE with other proteinase inhibitors or ligands present in the mixture. For example, PPE has higher affinity for α₂macroglobulin (α₂M) than for α₁PI, and when complexed with α₂M, PPM retains the ability to cleave small substrates. In the presence of α₂M, PPE binds α₂M and is protected from inhibition by α₁PI, and the complexation of PPE with α₂M can be measured by detecting the activity of PPE using SA³NA. To measure the inhibitory capacity of α₁PI in complex mixtures such as serum, two-fold serial dilutions of serum are incubated with a constant saturating concentration of PPE. The added PPE is bound by α₂M and α₁PI in the diluted serum dependant on their concentrations, the greater the concentration of serum, the greater the concentration of α₂M and α₁PI. Since there is more α₁PI in serum than α₂M, as serum is diluted, α₂M is diluted out, and in the absence of α₂M, PPE is bound and inhibited by α₁PI. The complexation of PPE with α₁PI can be measured by detecting the loss of activity of PPE using SA³NA. As serum is further diluted, α₁PI is also diluted out, and the loss of complexation of PPE with α₁PI can be measured by detecting the gain in activity of PPE using SA³NA. The plot of PPE activity versus serum dilution makes a V shaped curve, PPE activity first decreasing as serum is diluted, and then increasing as serum is further diluted. The nadir of PPE activity is used to calculate the precise concentration of active α₁PI in the mixture³⁹.

Induce receptor co-capping and cell motility: The procedures for inducing receptor capping have been described²². The cells of interest (monocytes, lymphocytes, neutrophils, or other blood cells, e.g. leukemic cells) are isolated from blood or tissue using standard techniques⁴⁰ and examined for reactivity with α₁PI.

To examine receptor capping, cells are incubated with active or modified α₁PI for 15 min in humidified 5% CO₂ at 37° C. Cells are applied to the sample chambers of a cytospin apparatus (Shandon Inc. Pittsburgh, Pa.), and slides are centrifuged at 850 rmp for 3 min. Slides are fixed by application of 50 μl 10% formalin to the sample chambers of the cytospin apparatus followed by an additional centrifugation at 850 rpm for 5 min. Slides are incubated for 90 min at 20° C. with fluorescently-labeled monoclonal antibodies having specificity for the receptors of interest and examined by microscopy.

Cell motility results from selective and sequential adherence and release produced by activation and deactivation of receptors^{41,42}, consequent polar segregation of related membrane proteins to the leading edge or trailing uropod, and both clockwise and counterclockwise propagation of Ca⁺⁺ waves which initiate from different locations in the cell⁴³. Thus, several aspects of the complex process may be quantitated. The most direct and most easily interpreted method for quantitating cell motility is the enumeration of adherent cells in response to a chemotactic agent such as α₁PI.

For detecting adherence, sterile coverslips are washed in endotoxin-free water, and to each coverslip is delivered various dilutions of active or modified α₁PI. Cells are subsequently delivered to the coverslips, mixed to uniformity with α₁PI, and incubated for 30 min in humidified 5% CO₂ at 37° C. without dehydration. After stringently washing the coverslips free of non-adherent cells, adherent cells are fixed by incubation for 10 min at 20° C. with 4% paraformaldehyde containing 2.5 μM of the nuclear staining fluorescent dye, acridine orange (3,6-bis[dimethylamino]acridine. Slides are examined by microscopy, and means and standard deviations are determined by counting adherent cells in at least three fields/coverslip.

Mobilize lymphoid-committed progenitor cells: In the nonobese diabetic/severe combined immunodeficiency (NOD/SCID) mouse model, bone marrow-engrafted human cells can be mobilized by G-CSF⁴⁴. This model is adapted to assess the capacity of α₁PI to mobilize human lymphoid- or myeloid-lineage cells, respectively.

NOD/SCID mice are housed under defined flora conditions in individually ventilated (HEPA-filtered air) sterile micro-isolator cages. Human chimeric mice are obtained after sublethal irradiation (375 cGy at 67 cGy/min) and injection of 2×10⁷ human cord blood mononuclear cells. Four to five weeks post transplantation, mobilization is performed by application of either G-CSF or α₁PI. For mobilization of myeloid-committed progenitors, mice receive daily subcutaneous injections of 300 μg/kg G-CSF (Filgrastim, Neupogen® or Neulasta®, Amgen, Inc.) in 250 μl of 0.9% NaCl, 5% fetal calf serum for 4-5 days. Alternatively, mice receive twice weekly infusion via the dorsal tail vein of inactive or modified α₁PI (39 mg/kg) at a rate of 0.08 ml/kg/minute. For mobilization of lymphoid-committed progenitors, mice receive twice weekly infusion via the dorsal tail vein of active or modified α₁PI (42 mg/kg) at a rate of 0.08 ml/kg/minute. Mice are asphyxiated with dry ice, peripheral blood is collected by cardiac aspiration into heparinized tubes, and bone marrow is harvested, and cells are flushed from femurs and tibias into single-cell suspensions. Peripheral blood and bone marrow cells are analyzed by flow cytometry for the presence of myeloid and lymphoid markers including CD34 CD38, CD10, CD11b, CD11c, CD13, CD14, CD19, CD3, CD4, CD8, CD45, CD184 (CXCR4), CD66, and HLE_{CS} (U.S. Pat. No. 6,858,400).

Bind anti-HIV-1 gp120: Active α₁PI is reactive with anti-HIV-1 gp120 antibodies in serum of HIV-1 patients at an epitope that is defined by the anti-gp120 monoclonal antibody 3F5 (hybridoma culture supernatant, 0085-P3F5-D5-F8) that reacts with an epitope near the gp120 C5 domain²⁵. Clone α70 (ICN Biochemicals, Aurora, Ohio) is reactive with the V3-loop of gp120, a domain that is identical to the HLE ligand inter-α-trypsin inhibitor⁴⁵ and is used as a negative control due to its lack of binding to α₁PI. Immune complexes are captured by incubating mixtures in wells of a microtiter plate pre-coated with chicken anti-human α₁PI IgG. Binding is detected using horse radish peroxidase-conjugated rabbit anti-mouse IgG followed by substrate, orthophenylene diamine HCl.

Facilitate HIV-1 infectivity: Primary non-syncytium inducing HIV-1 clinical isolates (Advanced Biotechnologies, Rivers Park, Ill.) are used to infect peripheral blood mononuclear cells maintained in wells of a 96 well tissue culture plate at 2×10⁶ cells/ml in RPMI-1640 containing 20% autologous serum and 10% IL-2 (Cellular Products, Buffalo, N.Y.). Prior to addition of HIV-1, cells are incubated with active α₁PI for 0 min or 60 min at 37° C., 5% CO₂. In vitro infectivity outcome is determined in triplicate by p24 accumulation or by RT activity as previously described⁴⁶. Cell counts and viability are determined at the final time point.

Treatment Outcome Measurements:

To determine the influence of α₁PI peptide treatment on elastase inhibitory capacity, individuals are monitored weekly for levels of active and inactive α₁PI in blood³⁹ (U.S. Pat. No. 6,887,678). Briefly, a constant amount of active site-titrated PPE is allowed to incubate with serial dilutions of serum for 2 min at 37° C. after which a PPE substrate is added. Determination of the molecules of substrate cleaved by residual, uninhibited PPE is used to calculate the molecules of active and inactive α₁PI in blood.

To determine the influence of α₁PI peptide treatment on inducing changes in levels of blood cell populations, treated individuals are monitored weekly for changes in complete blood count and differential, as well as for changes in specific subsets of blood cells such as CD4⁺ cells and HLE_{CS}⁺ cells using flow cytometry^{26,46} (U.S. Pat. No. 6,858,400). Briefly 100 μl of whole blood is incubated with a panel of fluorescently-labeled monoclonal antibodies approved by the FDA for medical diagnostics. These antibodies are selected to specifically recognize the cell receptors that uniquely identify the cell population of interest. Identification and enumeration of the cells in blood that are bound to the monoclonal antibodies is performed using flow cytometry.

To determine the influence of treatment on disease progression, individuals are monitored for the specific pathologic determinants of disease which are well known in the art for the various indications in HIV-1 disease. For example, in HIV-1 disease, individuals are monitored for changes in CD4⁺ lymphocyte levels and HIV levels^{26,46} as well as for signs of immune complex disease, emphysema, and respiratory illness related to α₁PI deficiency and autoimmunity.

### Dosage and Administration

A peptide or agent of the invention may be administered within a pharmaceutically-acceptable diluents, carrier, or excipient, in unit dosage form. Conventional pharmaceutical practice may be employed to provide suitable formulations or compositions to administer the compounds to patients suffering from a disease that is caused by excessive cell proliferation. Administration may begin before the patient is symptomatic. Any appropriate route of administration may be employed, for example, administration may be parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippiucourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for the peptides of the invention include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for a neoplastic disease or condition. The preferred dosage of a nucleobase oligomer of the invention is likely to depend on such variables as the type and extent of the disorder, the overall health status of the particular patient, the formulation of the compound excipients, and its route of administration.

With respect to a subject having a disease or disorder, an effective amount is sufficient to stabilize, slow, reduce, or reverse the disease or disorder being treated. With respect to treating HIV, an effective amount is sufficient to lead to normal blood levels of active alpha1PI. Generally, doses of compositions of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration.

Preferably, the peptides are administered at a dose between 1-100 μM.

In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or graft comprising appropriately transformed cells, etc., or parenteral routes.

Treatment Population

Active α₁PI promotes migration of lymphocytes and monocytic cells expressing HLE_{CS}²² (Examples and US 2008/0009442, incorporated by reference in its entirety herein). Inactive α₁PI promotes migration of neutrophils and cells expressing the LDL-receptor related protein, LRP^{47,48}, Treatment with active human α₁PI is indicated in individuals manifesting abnormal numbers of functional lymphocytes, monocytic cells, or dendritic cells such as in HIV-1 disease, stem cell transplantation, solid organ transplantation, autoimmune exacerbations, diabetes, leukemia, lymphoma, solid tumors, and, atherosclerosis. Treatment with inactive human α₁PI is indicated in individuals manifesting abnormal numbers of functional granulocytic, monocytic cells, dendritic, eosinophilic, or basophilic cells such as in microbial infection, neutropenia, and immunosuppressed patients. Treatment with α₁PI peptides is indicated in individuals manifesting α₁PI-reactive antibodies such as in HIV-1 disease. Treatment outcome is determined as described herein.

In certain preferred embodiments, the dosage of α₁PI peptides is determined by its capacity to saturate α₁PI-reactive antibodies as described herein. In preferred examples, individuals are injected with α₁PI peptides at the concentration that is equivalent to the detectable concentration of α₁PI-reactive antibodies. The frequency and length of treatment are determined by the disappearance of detectable α₁PI-reactive antibodies. In addition to being monitored for PPE inhibitory activity, α₁PI peptides are screened as described herein, for example for their capacity of to induce receptor capping and cell motility of lymphoid- and myeloid-lineage blood cells such as lymphocytes, neutrophils and stem cells.

### Therapy

Therapy may be provided wherever disease therapy is performed: at home, the doctor's office, a clinic, a hospital's outpatient department, or a hospital. Treatment generally begins at a hospital or clinic so that the doctor can observe the therapy's effects closely and make any adjustments that are needed. The duration of the therapy depends on the kind of disease being treated, the age and condition of the patient, the stage and type of the patient's disease, and how the patient's body responds to the treatment. Drug administration may be performed at different intervals (e.g., daily, weekly, or monthly). Therapy may be given in on-and-off cycles that include rest periods.

In certain preferred embodiments, the peptides are administered weekly. In other embodiments, the peptides are administered monthly.

As described above, if desired, treatment with a peptide of the invention may be combined with another agent (e.g., a therapeutic agent, antiretroviral therapy).

### Kits

The invention provides kits comprising a pharmaceutical composition comprising one or more peptides that block an interaction between α1 proteinase inhibitor and one or more molecules, and a pharmaceutically acceptable carrier, as described in any of the aspects herein. The kits can be used in any of the methods as described herein, for example in treating or preventing a disease or disorder in a subject. The invention also provides kits for use in treating a subject suffering from or susceptible to AIDS.

In certain embodiments, the kit comprises a sterile container, for example boxes, ampules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the peptides that block an interaction between α1 proteinase inhibitor and one or more molecules as described herein and their use in treating a disease or disorder. In other embodiments, the instructions include at least one of the following: methods for using the enclosed materials for the treatment or prevention of AIDS; precautions; warnings; indications; clinical or research studies; and/or references. The instructions may be printed directly on the container (when present), or as a label applied to the container, or as a separate sheet, pamphlet, card, or folder supplied in or with the container.

### EXAMPLES

### Example 1

### Monoclonal anti-gp120 binds human, but not chimpanzee α₁PI

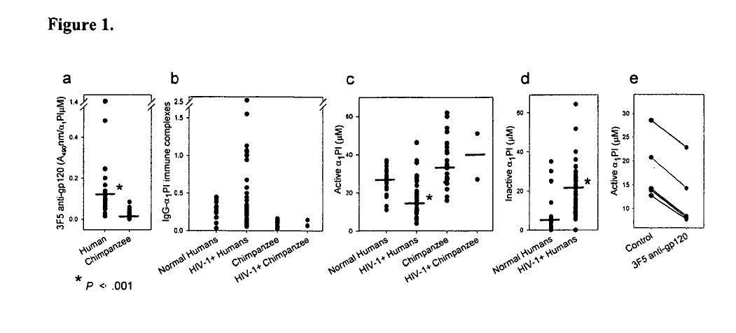

Two monoclonal antibodies (1C1 and 3F5) which bind a conformationally determined epitope near the C5 domain of gp120⁴⁹ were found to also bind human α₁PI²⁶. It was hypothesized that anti-gp120 mediated depletion of active α₁PI might be pathognomonic for HIV-1 AIDS. If true, chimpanzee α₁PI should differ from human α₁PI since HIV-1 infected chimpanzees survive infection and regain normal levels of CD4⁺ lymphocytes⁵⁰. Sequence comparison revealed that human α₁PI differs from chimpanzee α₁PI by one amino acid (aa 385) caused by a single nucleotide change (NCBI accession numbers BT019455 and XP_{—}522938), and this aa difference lies in the gp120-homologous region of α₁PI. To determine whether this sequence difference affects the binding of anti-gp120 to α₁PI, 20 human and 20 chimpanzee sera were compared. Both 1C1 (data not shown) and 3F5 exhibited 8- to 14-fold greater binding to human, than chimpanzee α₁PI in 6 repeat measurements (p<0.001) (FIG. 1a). Negative control monoclonal antibody α70 which reacts with the V3-loop of gp120 failed to bind human α₁PI (data not shown) consistent with previous findings²⁶. Serum α₁PI in two human subjects exhibited much greater affinity for 3F5 than that from other subjects, and this suggests the epitope of α₁PI recognized by 3F5 may be phenotypically determined, When these two subjects were omitted from the comparison, the statistical difference between binding of 3F5 to human or chimpanzee α₁PI was maintained (p<0.001).

To examine the relationship between lower CD4⁺ lymphocyte levels and lower active α₁PI levels in HIV-1 disease, blood from 38 HIV-1 infected patients was analyzed. Of these 38 patients, 29% had detectable IgG-α₁PI immune complexes, 89% were on antiretroviral therapy, and 60% had <500 HIV-1 RNA copies/ml (FIG. 1b). The number of patients exhibiting detectable IgG-α₁PI immune complexes in this study differs from a previous study of 68 HIV-1 patients in which 60% had detectable IgG-α₁PI immune complexes, 53% were on antiretroviral therapy (AZT only) and 16% had <500 HIV-1 RNA copies/ml²⁶. This reason for this difference may be related to the improved antiretroviral therapy in place today.

None of the sera from healthy chimpanzees, nor sera collected from 2 chimpanzees post-HIV-1 inoculation, had evidence of a detectable IgG-α₁PI immune complexes. The HIV-1 inoculated chimpanzees were confirmed to be HIV-1 infected, but had normal CD4⁺ lymphocytes⁵¹. In addition, despite the presence of anti-gp120, we found no evidence of IgG-α₁PI immune complexes in 10 rhesus macaques following immunization with simian/human immunodeficiency virus (SHIV 89.6) gp120 or gp140, or in 3 macaques infected with SHIV (data not shown). Extensive in vitro analyses failed to demonstrate bi-molecular complexes between gp120 and α₁PI (data not shown), and the absence of detection of IgG-α₁PI immune complexes in sera from HIV-1 infected chimpanzees suggests gp120 and α₁PI are not associated by aggregation in sera. These results suggest that IgG-α₁PI immune complexes are unique to HIV-1 disease in humans.

Consistent with evidence from a previous patient study, active α₁PI in the HIV-1 infected patients was significantly below normal (median 17 μM, p<0.001) (FIG. 1c) and inactive α₁PI was significantly above normal (median 19 μM, p<0.001) (FIG. 1d)²⁶. In contrast to humans, active α₁PI levels in sera collected from the 2 chimpanzees post-HIV-1 inoculation (39 μM) were not different from normal chimpanzee and human sera (p=0.810) (FIG. 1c).

To determine whether α₁PI becomes inactivated after complexing with the 3F5 anti-gp120 monoclonal antibody, 3F5 was incubated with sera samples from five healthy individuals. In comparison to untreated sera, α₁PI activity was significantly diminished to the same degree in all sera (mean difference=5.8±0.5 μM, p<0.001) (FIG. 1e).

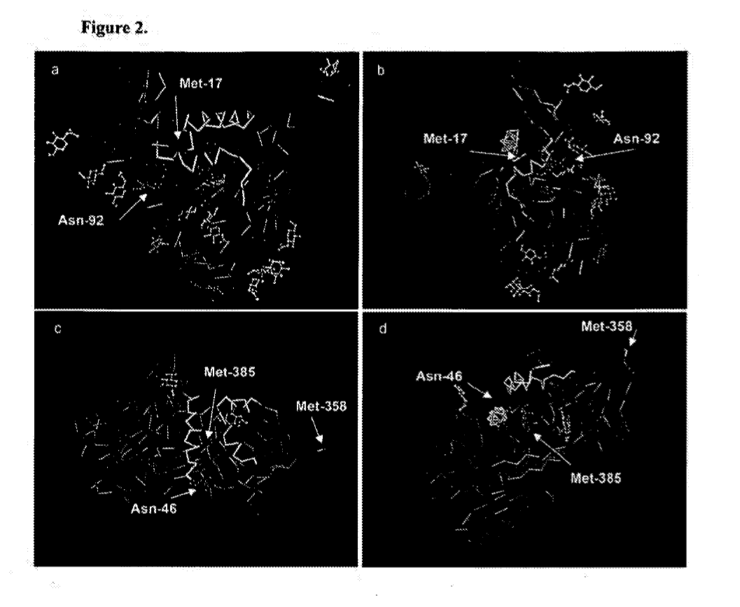

The gp120 epitope recognized by 1C1 and 3F5 is considered to be conformation-dependent⁴⁹. The gp120 peptide immunogen used to raise 1C1 and 3F5 (aa 300-321, GGGDMRDNWRSELYKYKVVK)⁵² contains both an α-helix (aa 306-313) and linear strand (aa 314-321) (FIG. 2a,b), but other epitope determinants of the antibodies are not known. In human α₁PI (FIG. 2c,d), the gp120-homologous sequence (aa 369-389, PFVFLMIDQNTKSPLFMGKVV) folds to form a two-stranded antiparallel β-sheet that lies at the base of a cleft (4 A° deep by 20 A° long by 5A ° wide) topped by two α-helices (aa 28-47 and 259-277) in a smaller, but similar configuration as the antigen-binding cleft of MHC (10 A° deep by 25 A° long by 10 A° wide)⁵³. At the far end of the first of these α-helices is the N-linked mannose-containing oligosaccharide that confers structural polymorphism to α₁PI (aa 46)¹⁵. In the center of the β-sheet that lies in the cleft, is M-385 which distinguishes human from chimpanzee α₁PI (V-385). The function of this cleft is not known but a sequence in the center of the β-sheet formation (aa 370-374 FVFLM) is homologous to the fusion domain of HIV-1 gp41, and this sequence has been implicated in binding HLE_{CS}^{22,54} and stimulating cell motility⁵⁵.

In α₁PI, the sequence GKVV (aa 386-389) lies within 5 A° of M-385, N-46, and the N-linked oligosaccharide in a space occupying 5 A° by 5 A° by 5 A°. In gp120, in the same relative orientation as in α₁PI, the sequence YKVV (aa 315-318) lies within 5 A° of M-17 and 8 A° of N-92 and the N-linked mannose-containing oligosaccharide⁵⁶ in a space occupying 5 A° by 5 A° by 8 A°. Evidence that α₁PI polymorphisms may influence 3F5 binding (FIG. 2a) suggests the polymorphism-determining N-46 oligosaccharide participates in 3F5 recognition of α₁PI. Thus, the 3F5 conformational epitope is suggested by these analyses to occupy a 5 A° by 5 A° by 8 A° space including KVV, M, N, and the N-linked oligosaccharide. This proposed conformational epitope is consistent with previously characterized antigen configurations that contain oligosaccharide determinants⁵⁷ as well as with results demonstrating that gp120 N-92 is invariant⁵⁸.

### Example 2

### Active α₁PI is rate limiting for CD4⁺ lymphocytes in HIV-1 disease.

Of the 36 patients included in the study population, 23 were below 500 and 13 were above 500 HIV RNA copies/ml at the time of blood collection. All patients were measured for CD4, CXCR4, CCR5, SDF-1 levels, active and inactive α₁PI. Only 28 of these patients were additionally measured for HLE_{CS}. Neither CXCR4 nor CCR5 were found to correlate individually or in combination with any parameters of disease being investigated in these patients. Eleven of the 38 HIV-1 patients had active liver disease as defined by detectable Hepatitis B or C, or elevated liver enzymes. HIV-1 patients with liver disease were not different from patients without liver disease in active α₁PI (p=0.95), total α₁PI (p=0.79), CXCR4 (p=0.63), or CCR5 (p=0.9), but exhibited significantly higher SDF-1 (p<0.001), HLE_{CS}⁺ lymphocytes (p<0.001), and CD4⁺ lymphocytes (p=0.04).

In the 23 patients with <500 HIV-1 RNA copies/ml, higher CD4⁺ lymphocyte levels were correlated with higher active α₁PI concentration (r²=0.927) and lower inactive α₁PI concentration (r²=0.946) (FIG. 3). Prediction of CD4 levels from active α₁PI levels with 95% confidence had a standard error of 151 cells/μl, and prediction from inactive α₁PI levels with 95% confidence had a standard error of 105 cells/μl. Of these 23 patients, only 16 had been additionally measured for HLE_{CS}. As in Healthy individuals, lower HLE_{CS}⁺ lymphocytes was itself not correlated with higher CD4⁺ lymphocytes, but in combination with higher active α₁PI was significantly correlated (p=0.01). That CD4⁺ lymphocyte levels could be predicted by active α₁PI alone with such a high degree of accuracy in patients controlling their viral load suggests that, unlike the normal population, active α₁PI is rate limiting for CD4⁺ lymphocyte levels in HIV-1 disease. In patients with >500 HIV RNA copies/ml there was no relationship between CD4⁺ lymphocyte levels and active or inactive α₁PI (FIG. 3), and this suggests either HIV-1 itself, or other host processes had contributed to disrupting the regulation of CD4⁺ lymphocyte levels.

### Example 3

### α₁PI Augmentation Therapy in HIV-1 Infected Patients

The number of CD4⁺ T lymphocytes in patients with <500 HIV-1 RNA copies/ml is controlled by their circulating concentration of α₁PI (Example 3). These patients have below normal levels of circulating α₁PI²⁶. Approximately 10% clinic patients in New York City who have <500 HIV-1 RNA copies/ml also have <200 CD4 cells/μl, and these patients benefit from α₁PI augmentation by increasing their CD4⁺ T lymphocyte numbers. Treatment of HIV-1 infected patients with α₁PI augmentation is indicated in patients who are simultaneously receiving one or a combination of the four currently known classes, nucleoside reverse transcriptase inhibitors non-nucleoside reverse transcriptase inhibitors, HIV-1 aspartyl protease inhibitors, and fusion inhibitors.

Three patients with <500 HIV-1 RNA copies/ml and <300 CD4 cells/μl who were receiving antiretroviral therapy were placed on ZEMAIRA α₁PI augmentation therapy. Patients received weekly infusions of Zemaira® at 120 mg/kg. Treatment outcome was monitored as described herein. Specifically, patients receiving ZEMAIRA were monitored weekly for changes in active and inactive α₁PI levels as well as for CD4⁺ T lymphocytes and other subsets of circulating blood cells. Patients were also monitored for changes in HIV-1 RNA copies/ml, LDL, HDL, cholesterol, triglycerides, and the occurrence of infections designated by the CDC as parameters of HIV-1 disease progression⁵⁹. To determine possible adverse effects of immune complex disease, individuals were monitored for the presence of antibodies reactive with α₁PI as well as for the occurrence of glomerulonephritis by measuring either proteinuria or serum creatinine levels^{26,60}.

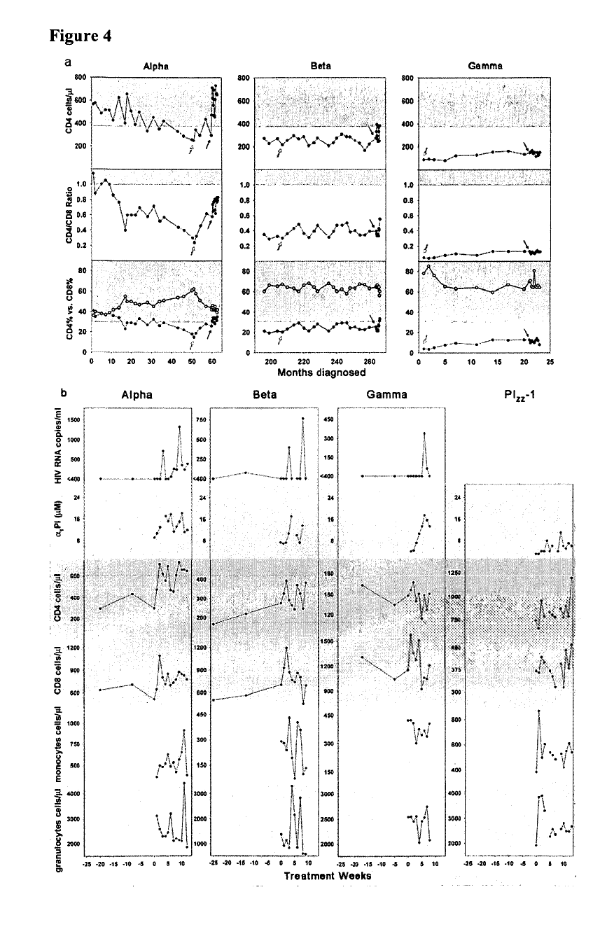

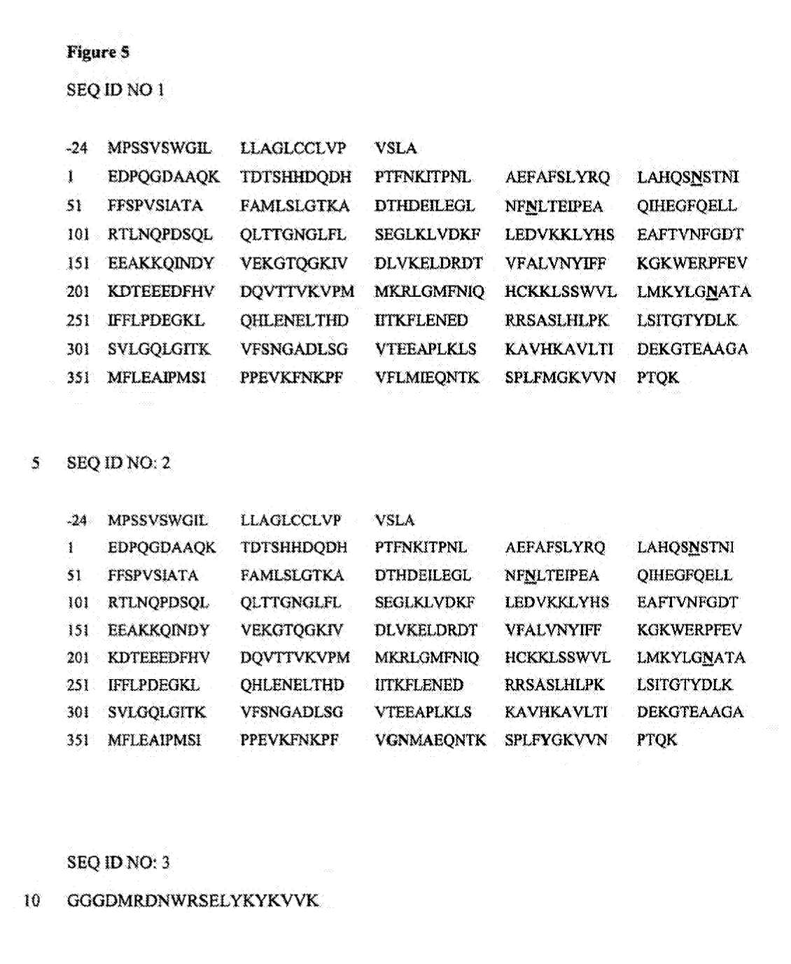

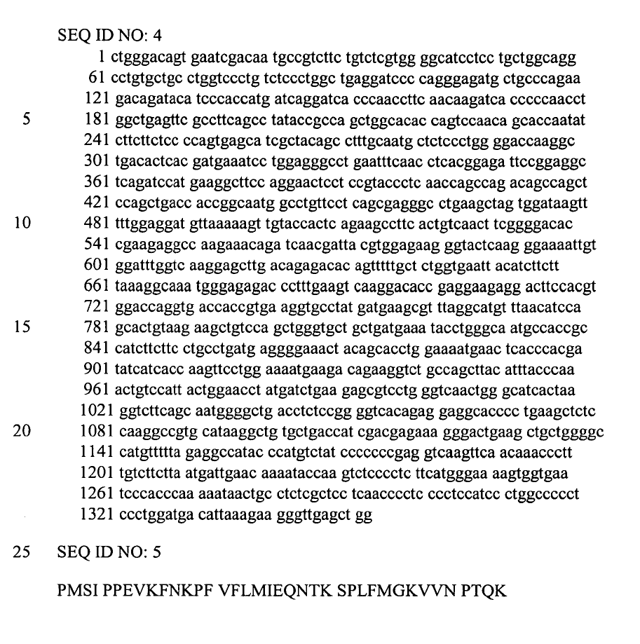

After 2 weeks of therapy, 2 of 3 patients achieved a normal number of CD4⁺ lymphocytes with increases from 297 to 710 and from 276 to 393 cells/μl, respectively (FIG. 4). In addition an HIV-1 uninfected patient with normal numbers of CD4⁺ lymphocytes and a diagnosis of emphysema in the context of genetic α₁PI deficiency received weekly infusions of 60 mg/kg α₁PI augmentation. This patient increased from 743 to 954 CD4⁺ cell/μl.

### Example 4

### α₁PI Augmentation Therapy in HIV-1 Infected Patients Using α₁PI Peptides

Antibodies that recognize HIV-1 are the only diagnostic marker of infectivity. The presence of an anti-gp120 antibody that also binds α₁PI has been detected in 90% HIV-1 infected individuals²⁶, and this antibody inactivates and produces deficient levels of α₁PI. Anti-gp120 does not bind chimpanzee α₁PI which differs from human α₁PI by a single amino acid (aa 385) (Example 1). To therapeutically augment α₁PI in HIV-1 infected individuals, it is desirable to use α₁PI peptides that have higher affinity for anti-gp120 than human α₁PI thereby releasing native α₁PI from the antibodies and blocking these antibodies from further binding to newly synthesized native α₁PI. To maintain HIV-1 immunity, it is desirable to use α₁PI peptides that have lower affinity for anti-gp120 than HIV-1 gp120.

To produce α₁PI peptides that have higher affinity for anti-gp120 than α₁PI and lower affinity for anti-gp120 than HIV-1 gp120, peptides are derived from modification of the α₁PI sequence (aa 369-389, PFVFLMIDQNTKSPLFMGKVV) as described herein. Modification of α₁PI peptides is for the purpose of increasing their binding to antibodies that recognize the HIV-1 gp120 epitope (aa 300-321, GGGDMRDNWRSELYKYKVVK). An α₁PI peptide with such substitutions includes aa 372 (Phe to Gly), aa 373 (Leu to Asp), aa 375 (Ile to Arg), and aa 385 (Met to Tyr). The full length α₁PI representing such changes is designated α₁PI.β.F372G.L373D.I375R.M385Y (α₁PI.β). The α₁PI.β sequence with aa changes represented in bold underlined letters is as follows:

| | |
|---|---|
| -24 | MPSSVSWGIL LLAGLCCLVP VSLA |
| 1 | EDPQGDAAQK TDTSHHDQDH PTFNKITPNL AEFAFSLYRQ |
| | LAHQSNSTNI |
| 51 | FFSPVSIATA FAMLSLGTKA DTHDEILEGL NFNLTEIPEA |
| | QIHEGFQELL |
| 101 | RTLNQPDSQL QLTTGNGLFL SEGLKLVDKF LEDVICKLYH |
| | SEAFTVNFGDT |
| 151 | EEAKKQINDY VEKGTQGKIV DLVKELDRDT VFALVNYIFF |
| | KGKWERPFEV |
| 201 | KDTEEEDFHV DQVTTVKVPM MKRLGMFNIQ HCKKLSSWVL |
| | LMKYLGNATA |
| 251 | IFFLPDEGKL QHLENELTHD IITKFLENED RRSASLHLPK |
| | LSITGTYDLK |
| 301 | SVLGQLGITK VFSNGADLSG VTEEAPLKLS KAVHKAVLTI |
| | DEKGTEAAGA |
| 351 | MFLEAIPMSI PPEVKFNKPF VGDMRDQNTK SPLFYGKVVN |
| | PTQK |

The half life of therapeutic α₁PI is 4.5 days and thus patients require weekly treatment. The recommended therapeutic dose is 60 mg/kg/week which raises serum α₁PI to the level of 11 μM in individuals with genetic α₁PI deficiency. The specific activity of Zemaira® is 70%, where specific activity is defined as inhibition of PPE³⁹. Thus, the dose of Zemaira® α₁PI that elevates serum α₁PI to acceptable levels may be stated as 42 mg/kg active α₁PI/week or 3 g/week for a 70 kg adult. However, the half life of IgG is 4 weeks, and the average concentration of IgG is 10 mg/ml or 67 μM. In a gross overestimation, if 10% of a patient's serum IgG recognized α₁PI, this would mean 6.7 μM IgG would need to be displaced from α₁PI by 6.7 μM α₁PI peptides once every 4 weeks. The molecular mass of the α₁PI.β peptide is 2277 daltons, and 7 μM α₁PI.β represents 15 μg/ml or 15 mg/L. Since the volume of blood in a healthy individual is approximately 1/11 body weight, a 70 kg adult has a blood volume of approximately 5 L. Thus, to saturate 10% of serum IgG in a 70 kg adult, the recommended dose of α₁PI.β is 75 mg/5 L/4 weeks or 5 mg/week. For comparison, the recommended dose of Lantus® recombinant insulin (6063 daltons, Aventis Pharma) is as much as 3.6 mg/ml/day with a half life of 12 hrs.

Treatment of HIV-1 patients with <500 HIV-1 RNA copies/ml and <500 CD4 cells/μl who are receiving antiretroviral therapy consists of an injection of 74 μg/kg/week α₁PI.β or 5 mg/70 kg/week with a target blood threshold of 7 μM α₁PI.β. Treatment of HIV-1 infected patients with α₁PI.β is indicated in patients who are simultaneously receiving one or a combination of the four currently known classes, nucleoside reverse transcriptase inhibitors, non-nucleoside reverse transcriptase inhibitors, HIV-1 aspartyl protease inhibitors, and fusion inhibitors, Treatment outcome is monitored as described herein. Specifically, patients receiving α₁PI.β are monitored weekly for changes in active and inactive α₁PI levels as well as for CD4⁺ T lymphocytes and other subsets of circulating blood cells. Patients are also monitored for changes in HIV-1 RNA copies/ml, LDL, HDL, cholesterol, triglycerides, and the occurrence of infections designated by the CDC as parameters of HIV-1 disease progression⁵⁹. To determine possible adverse effects of immune complex disease, individuals are monitored for the presence of antibodies reactive with α₁PI as well as for the occurrence of glomerulonephritis by measuring either proteinuria or serum creatinine levels^{26,60}.

### REFERENCES

1. Berninger, R. W. Alpha 1-antitrypsin. J. Med. 16, 23-99 (1986).

2. Brantyl, M. L. et al. Use of a highly purified alpha 1-antitrypsin standard to establish ranges for the common normal and deficient alpha 1-antitrypsin phenotypes. Chest 100, 703-708 (1991).

3. Bristow, C. L, di Meo, F. & Arnold, R. R. Specific activity of a1proteinase inhibitor and a2macroglobulin in human serum: Application to insulin-dependent diabetes mellitus. Clin. Immunol. Immunopathol. 89, 247-259 (1998).

4. Kushner, I. The phenomenon of the acute phase response. Ann. N.Y. Acad. Sci. 389, 39-47 (1982).

5. OMIM. Online Mendelian Inheritance in Man, OMIM™. McKusick-Nathans Institute for Genetic Medicine, Johns Hopkins University (Baltimore, Md.) and National Center for Biotechnology Information, National Library of Medicine (Bethesda, Md.). 2000.

RefType: Date File

6. Berninger, R. W. Alpha 1-antitrypsin. J. Med. 16, 23-99 (1985).

7. Bristow, C. L., Mercatante, D. R. & Kole, R. HIV-1 preferentially binds receptors co-patched with cells surface elastase. Blood 102, 4479-4486 (2003).

8. Tavor, S. et al. Motility, proliferation and egress to the circulation of human AML cells in transplanted NOD/SCID mice are elastase dependent. Blood 106, 2120-2127 (2005).

9. Gullberg, U. et al. Carboxyl-terminal prodomain-deleted human leukocyte elastase and cathepsin G are efficiently targeted to granules and enzymatically activated in the rat basophilic/mast cell line RBL. J. Biol. Chem. 270, 12912-12918 (1995).

10. Garwicz, D., Lennartsson, A., Jacobsen, S. E. W., Gullberg, U. & Lindmark, A. Biosynthetic profiles of neutrophil serine proteases in a human bone marrow-derived cellular myeloid differentiation model. Haematologica 90, 38-44 (2005).

11. Wolf, K., Muller, R., Borgmann, S., Brocker, E. B. & Friedl, P. Amoeboid shape change and contact guidance: T-lymphocyte crawling through fibrilar collagen is independent of matrix remodeling by MMPs and other proteases. Blood 102, 3262-3269 (2003).

12. Lapidot, T. & Petit, I. Current understanding of stem cell mobilization: The roles of chemokines, proteolytic enzymes, adhesion molecules, cytokines, and stromal cells. Exp. Hematol. 30, 973-981 (2002).

13. Cepinskas, G., Sandig, M. & Kvietys, P. R. PAF-induced elastase-dependent neutrophil transendothelial migration is associated with the mobilization of elastase to the neutrophil surface and localization to the migrating front. J. Cell Science 112, 1937-1945 (1999).

14. Nukiwa, T. et al. Identification of a second mutation in the protein-coding sequence of the Z type alpha 1-antitrypsin gene. J. Biol. Chem. 261, 15989-15994 (1986).

15. Jeppsson, J. O., Lilja, H. & Johansson, M. Isolation and characterization of two minor fractions of alpha 1-antitrypsin by high-performance liquid chromatographic chromatofocusing. J. Chromatogr. 327, 173-177 (1985).

16. Brantly, M. L. et al. Use of a highly purified alpha 1-antitrypsin standard to establish ranges for the common normal and deficient alpha 1-antitrypsin phenotypes. Chest 100, 703-708 (1991).

17. Sifers, R. N, Brashears-Macatee, S., Kidd, V. J., Muensch, H. & Woo, S. L. A frameshift mutation results in a truncated alpha 1-antitrypsin that is retained within the rough endoplasmic reticulum. Journal of Biological Chemistry 263, 7330-7335 (1988).

18. Perkins, S. J. et al. Secondary structure changes stabilize the reactive-centre cleaved form of SERPINs. J. Mol. Biol. 228, 1235-1254 (1992).

19. Mellet, P., Boudier, C., Mely, Y. & Bieth, J. G. Stopped Flow Fluorescence Energy Transfer Measurement of the Rate Constants Describing the Reversible Formation and the Irreversible Rearrangement of the Elastase-alpha 1-Proteinase Inhibitor Complex. Journal of Biological Chemistry 273, 9119-9123 (1998).

20. Elliott, P. R., Pei, X. Y., Dafforn, T. R. & Lomas, D. A. Topography of a 2.0 A structure of alpha1-antitrypsin reveals targets for rational drug design to prevent conformational disease [In Process Citation]. Protein Sci 9, 1274-1281 (2000).

21. Joslin, G. et al. The serpin-enzyme complex (SEC) receptor mediates the neutrophil chemotactic effect of a-1 antitrypsin-elastase complexes and amyloid-b peptide. J. Clin. Invest. 90, 1150-1154 (1992).

22. Bristow, C. L., Mercatante, D. R. & Kole, R. HIV-1 preferentially binds receptors co-patched with cell surface elastase. Blood 102, 4479-4486 (2003).

23. Banda, M. J., Rice, A. G., Griffin, G. L. & Senior, R. M. a1-proteinase inhibitor is a neutrophil chemoattractant after proteolytic inactivation by macrophage elastase. J. Biol. Chem. 263, 4481-4489 (1988).

24. Moore, J. P., Sattentau, Q. E., Wyatt, R. & Sodroski, J. Probing the structure of the human immunodeficiency virus surface glycoprotein gp120 with a panel of monoclonal antibodies. J. Virol. 68, 469-485 (1994).

25. Moore, J. P., Cao, Y., Ho, D. D. & Koup, R. A. Development of the anti-gp120 antibody responses during seroconversion to human immunodeficiency virus type 1. J. Virol. 68, 5142-5144 (1994).

26. Bristow, C. L., Patel, H. & Arnold, R. R. Self antigen prognostic for human immunodeficiency virus disease progression. Clin Diagn. Lab. Immunol. 8, 937-942 (2001).

27. Haynes, B. F. et al. Cardiolipin Polyspecific Autoreactivity in Two Broadly Neutralizing HIV-1 Antibodies. Science 308, 1906-1908 (2005).

28. Courtney, M. et al. High-level production of biologically active human a1-antitrypsin in Escherichia coli. Proc Natl Acad Sci; USA 81, 669-673 (1984).

29. Terashima, M. et al. Production of functional human a1-antitrypsin by plant cell culture. Appl Microbiol Biotechnol 52, 516-523 (1999).

30. Jean, F. et al. a1-antitrypsin Portland, a bioengineered serpin highly selective for furin: Application as an antipathogenic agent. Proc Natl Acad Sci; USA 95, 7293-7298 (1998).

31. Parfrey, H. et al. Targeting a surface cavity of a1-antitrypsin to prevent conformational disease. J. Biol. Chem. 278, 33060-33066 (2003).

32. Current Protocols in Molecular Biology. Greene Publishing Associates and Wiley-Intersciences, New York, (2002).

33. Fields, G. B. & Noble, R. L. Solid phase peptide synthesis utilizing 9-fluorenylmethoxycarbonyl amino acids. Int J Pept Protein Res. 35, 161-214 (1990).

34. Poller W., Willnow, T. E., Hilpert, J. & Herz, J. Differential recognition of alpha 1-antitrypsin-elastase and alpha 1-antichymotrypsin-cathepsin-G complexes by the low density lipoprotein receptor-related protein. J. Biol. Chem. 270, 2841-2845 (1995).

35. Rodenburg, K. W., Kjoller, J., Petersen, H. H. & Andreasen, P. A. Binding of urokinase-type plasminogen activator-plasminogen activator inhibitor-1 complex to the endocytosis receptors alpha2-macroglobulin receptor/low-density lipoprotein receptor-related protein and very-low-density lipoprotein receptor involves basic residues in the inhibitor. Biochemical Journal 329, 55-63 (1998).

36. Janciauskiene, S., Wright, H. T. & Lindgren, S. Atherogenic properties of human monocytes induced by the carboxyl terminal proteolytic fragment of alpha-1-antitrypsin. Atherosclerosis 147, 263-275 (1999).

37. Janciauskiene, S. & Lindgren, S. Effects of fibrillar C-terminal fragment of cleaved alpha1-antitrypsin on cholesterol homeostasis in HepG2 cells. Hepatology 29, 434-442 (1999).

38. Dichtl, W. et al. The Carboxyl-Terminal Fragment of [alpha]1-Antitrypsin Is Present in Atherosclerotic Plaques and Regulates Inflammatory Transcription Factors in Primary Human Monocytes. Molecular Cell Biology Research Communications 4, 50-61 (2000).

39. Bristow, C. L., di Meo, F. & Arnold, R. R. Specific activity of a1proteinase inhibitor and a2macroglobulin in human serum: Application to insulin-dependent diabetes mellitus. Clin. Immunol. Immunopathol. 89, 247-259 (1998).

40. Messmer, D. et al. Endogenously expressed nef uncouples cytokine and chemokine production from membrane phenotypic maturation in dendritic cells. J. Immunol. 169, 4172-4182 (2002).

41. Wright, S. D. & Meyer, B. C. Phorbol esters cause sequential activation and deactivation of complement receptors on polymorphonuclear leukocytes. J. Immunol. 136, 1759-1764 (1986).

42. Ali, H., Tomhave, E. D., Richardson, R. M., Haribabu, B. & Snyderman, R. Thrombin primes responsiveness of selective chemoattractant receptors at a site distal to G protein activation. J. Biol. Chem. 271, 3200-3206 (1996).

43. Kindzelskii, A. L. & Petty, H. R. Intracellular Calcium Waves Accompany Neutrophil Polarization, Formylmethionylleucylphenylalanine Stimulation, and Phagocytosis: A High Speed Microscopy Study. J. Immunol. 170, 64-72 (2003).

44. Petit, I. et al. G-CSF induces stem cell mobilization by decreasing bone marrow SDF-1 and up-regulating CXCR4. Nature Immunol 3, 687-694 (2002).

45. Pratt, C. W., Roche, P. A. & Pizzo, S. V. The role of inter-a-trypsin inhibitor and other proteinase inhibitors in the plasma clearance of neutrophil elastase and plasmin. Arch. Biochem. Biophys. 258, 591-599 (1987).

46. Bristow, C. L. Slow human immunodeficiency virus (HIV) infectivity correlated with low HIV coreceptor levels. Clin. Diagn. Lab. Immunol. 8, 932-936 (2001).

47. Kounnas, M. Z., Church, F. C., Argraves, W. S. & Strickland, D. K. Cellular internalization and degradation of antithrombin III-thrombin, heparin cofactor II-thrombin, and alpha 1-antitrypsin-trypsin complexes is mediated by the low density lipoprotein receptor-related protein. J. Biol. Chem. 271, 6523-6529 (1996).

48. Weaver, A. M., Hussaini, I. M., Mazar, A., Henkin, J. & Gonias, S. L. Embyronic Fibroblasts That Are Genetically Deficient in Low Density Lipoprotein Receptor-related Protein Demonstrate Increased Activity of the Urokinase Receptor System and Accelerated Migration on Vitronectin, Journal of Biological Chemistry 272, 14372-14379 (1997).

49. Moore, J. P., Sattentau, Q. E., Wyatt, R. & Sodroski, J. Probing the structure of the human immunodeficiency virus surface glycoprotein gp120 with a panel of monoclonal antibodies. J. Virol. 68, 469-484 (1994).

50. Rutjens, E. B.-J., Verschoor, E., Bogers, W., Koopman, G. & Heeney, J. Lentivirus infections and mechanisms of disease resistance in chimpanzees. Front Biosci. 8, d1134-d1145 (2003).

51. Girard, M. et al. Genital infection of female chimpanzees with human immunodeficiency virus type 1. AIDS Res Hum Retrovirus 14, 1357-1367 (1998).

52. Ratner, L. et al. Complete nucleotide sequence of the AIDS virus, HTLV-III. Nature 313, 277-284 (1985).

53. Bjorkman, P. J. et al. Structure of the human class I histocompatibility antigen, HLA-A2. Nature 329, 506-512 (1987).

54. Bristow, C. L., Fiscus, S. A., Flood, P. M. & Arnold, R. R. Inhibition of HIV-1 by modification of a host membrane protease. Int. Immunol. 7, 239-249 (1995).

55. Joslin, G., Fallon, R. J., Bullock, J., Adams, S. P. & Perlmutter, D. H. The SEC receptor recognizes a pentapeptide neodomain of alpha-1-antitrypsin-protease. J. Biol. Chem. 266, 11282-11288 (1991).

56. Leonard, C. K. et al. Assignment of intrachain disulfide bonds and characterization of potential glycosylation. J. Biol. Chem. 265, 10373-10382 (1987).

57. Cygler, M., Rose, D. R. & Bundle, D. R. Recognition of a cell-surface oligosaccharide of pathogenic Salmonella by an antibody Fab fragment. Science 253, 442-445 (1991).

58. Wei, X. et al. Antibody neutralization and escape by HIV-1. Nature 422, 307-312 (2003).

59. Castro, K. G. et al. 1993 revised classification system for HIV infection and expanded surveillance case definition for AIDS among adolescents and adults. Morbid. Mortal. Weekly Rep. 41, 1-19 (1992).

60. Virella, G. et al. Soluble immune complexes in patients with Diabetes Mellitus: Detection and pathological significance. Diabetologia 21, 184-191 (1981).

1.-75. (canceled)

76. A method for increasing the number of circulating CD4+ lymphocytes in an HIV-1-infected human subject comprising administering to a subject in need of such treatment a pharmaceutical composition comprising an amount of an active α1 Protease Inhibitor (α1 PI) effective to increase the number of circulating CD4+ lymphocytes in said subject, wherein said subject has less than 500 circulating CD4+ lymphocytes per μl and is receiving antiretroviral therapy.

77. The method of claim 76 wherein said subject has less than 500 copies of HIV-1 RNA per ml.

78. The method of claim 77 wherein said patient is receiving antiretroviral therapy.

79. A method for determining the number of CD4+ lymphocytes in the blood of an HIV-1 infected human subject comprising the steps of:
determining the amount of active α1 PI in the serum of the human subject by assaying for α1 PI activity;
wherein the number of CD4+ lymphocytes in the subject's serum is proportional to the amount of active α1 PI present in said subject's serum and said subject has less than 500 HIV-1 RNA copies/ml; and
administering a composition comprising an amount of active α1 PI effective to increase the number of CD4+ lymphocytes in said subject's serum.

80. The method of claim 79 wherein the activity of active α1 PI is determined by an assay method for α1 PI selected from the group consisting of elastase inhibition, induction of receptor co-capping and cell motility, mobilization of lymphoid-committed progenitor cells, the ability to bind to HIV-1 anti-gp120 antibodies and facilitation of HIV-1 infectivity.

81. A method for identifying active α1 PI peptides that have a higher affinity for the HIV-1 gp120 monoclonal antibody than the active human α1 PI peptide comprising the steps of:
providing a test peptide and a control peptide;
incubating the test peptide and the control peptide in serum obtained from an HIV-1-infected subject for a time and at a temperature effective for the peptides and gp120 antibodies present in the serum to interact, and
assaying for active α1 PI activity, wherein an increase in active α₁ PI activity using the test peptide compared to the control peptide indicates that the test peptide has a higher affinity for the HIV-1 gp120 monoclonal antibody than the human α1 PI peptide.

82. The method of claim 81 wherein the activity α1 PI activity is assayed by an assay method selected from the group consisting of elastase inhibition, induction of receptor co-capping and cell motility, mobilization of lymphoid-committed progenitor cells, the ability to bind to HIV-1 gp120 antibodies and facilitation of HIV-1 infectivity.

83. A method for activating α1 PI in the serum of a patient infected with HIV-1 and having HIV-1 anti-gp120 antibodies comprising administering to a patient in need of such treatment a composition comprising an α1 PI peptide having higher affinity for HIV-1 anti-gp120 monoclonal antibody than human α1 PI for a time and at a temperature effective for the peptide to block the interaction between HIV-1 gp120 antibodies and the α1 PI present in said patient's serum, thereby activating the α1 PI.

84. The method of claim 83 wherein activating the α1 PI in the patient's serum leads to an increase in α1 PI activity.

85. The method of claim 84 wherein activating the α1 PI leads to lymphocyte renewal.

86. The method of claim 85 wherein said patient has less than 500 HIV-1 RNA copies/ml.

87. The method of claim 86 wherein said patient has less than 500 CD4+ lymphocytes/μl.

88. A method for determining the number of CD4+ lymphocytes in the blood of an HIV-1 infected human subject comprising the steps of:
providing a serum sample from said patient,
determining the amount of active α1 PI in the serum of said human subject by assaying for α1 PI activity;
wherein the number of CD4+ lymphocytes in said subject's serum is proportional to the amount of active α1 PI present in said subject's serum and said subject has less than 500 HIV-1 RNA copies/ml; and
administering a composition comprising an amount of active α1 PI effective to increase the number of CD4+ lymphocytes in said patient's serum.
